SEQUENCE LISTING (6 sequences)

SEQ ID NO: 1 - DNA, 214 bp, Artificial Sequence
  P21 minimal promoter
tgctggaactcggccaggctcagctggctcggcgctgggcagccaggagcctgggccccggggagggcggtcccgggcggcgcggtgggccgagcgcgggtcccgcctccttgaggcgggcccgggcggggcggttgtatatcagggccgcgctgagctgcgccagctgaggtgtgagcagctgccgaagtcagttccttgtggagccaagctt

SEQ ID NO: 2 - DNA, 37 bp, Artificial Sequence
  PCR forward primer
taacggaagatcttgctggaactcggccaggctcagc

SEQ ID NO: 3 - DNA, 44 bp, Artificial Sequence
  PCR reverse primer
actcggtggtaccaagcttggctccacaaggaactgacttcggc

SEQ ID NO: 4 - DNA, 5499 bp, Artificial Sequence
  Plasmid p21m-BLA
gacggatcgggagatcttgctggaactcggccaggctcagctggctcggcgctgggcagccaggagcctgggccccggggagggcggtcccgggcggcgcggtgggccgagcgcgggtcccgcctccttgaggcgggcccgggcggggcggttgtatatcagggccgcgctgagctgcgccagctgaggtgtgagcagctgccgaagtcagttccttgtggagccaagcttggtaccaccatggacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggkttattgctgataaatctggasccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 5 - DNA, 24 bp, Artificial Sequence
  PCR forward primer
cgcgcacatttccccgaaaagtgc

SEQ ID NO: 6 - DNA, 27 bp, Artificial Sequence
  PCR reverse primer
gcatttaggtgacactatagaataggg

(19) United States
(12) Patent Application Publication
(10) Pub. No.: US 2008/0057529 A1
(43) Pub. Date: Mar. 6, 2008

(54) Method for Identifying Histone Deacetylase Inhibitors

(76) Inventors: Michele Pallaoro, Rome (IT); Paola Gallinari, Rome (IT); Sergio Altamura, Rome (IT); Christian Steinkuhler, Rome (IT)

Correspondence Address: MERCK AND CO., INC, P O BOX 2000, RAHWAY, NJ, 07065-0907, US

(21) Appl. No.: 10/582,621

(22) PCT Filed: Dec. 10, 2004
(86) PCT No.: PCT/EP04/14159

§ 371 Date: Jun. 12, 2006

Related U.S. Application Data
(60) Provisional application No. 60/530,540, filed on Dec. 18, 2003.

(51) Int. Cl. C12Q 1/02; C12N 15/00; C12N 5/06

(52) U.S. Cl. 435/29; 435/320.1; 435/325

(57) ABSTRACT

A method for identifying analytes which are histone deacetylase (HDAC) inhibitors is described. The method provides cells comprising a reporter gene operably linked to a transcription regulatory sequence, which includes nucleotide sequences responsive to a known HDAC inhibitor or subtype, stably integrated into the genome of the cells. A culture of the cells is incubated in medium which contains an analyte and the culture monitored for expression of the reporter gene. Analytes which have HDAC inhibitor activity induce expression of the reporter gene. In particular embodiments, the transcription regulatory region is a sequence of the p21WAF1/CIP1 transcription regulatory sequence which is responsive to a known HDAC inhibitor but not responsive to p53.

### BACKGROUND OF THE INVENTION

(1) Field of the Invention

The present invention relates to a cell-based method for identifying histone acetylase (HDAC) inhibitors. The method is particularly suitable for high throughput screening.

(2) Description of Related Art

The reversible acetylation of lysine amino groups in nuclear histones is one of the important mechanisms by which gene expression is regulated. Histones are highly conserved basic proteins which associate with nuclear DNA to form a compact complex called chromatin. The core histones H2A, H2B, H3, and H4 associate to form a protein core which DNA then winds around, with the basic amino acids of the histones interacting with the negatively charged phosphate groups of the DNA. About 146 base pairs of DNA wrap around a histone core to make up a nucleosome, the repeating structural motif of chromatin.

In normal cells, the competing activities of two enzyme classes, the histone acetyl transferases (HATs) and histone deacetylases (HDACs) result in cell-specific patterns of gene expression. Acetylation neutralizes the positive charge of the lysine side chains of the histones and is thought to affect chromatin structure. Taunton et al., Science, 272: 408-411 (1996), showed that hyperacetylation enhances access of transcription factors to chromatin. In contrast, underacetylated histone H4 has been found in transcriptionally silent regions of the genome. Thus, it appears that hyperacetylated chromatin regions usually correlate with active gene transcription whereas hypoacetylated chromatin regions of the genome are usually poorly transcribed. All known HDAC inhibitors induce cell-cycle arrest at G1/S or G2/M and, depending on the cell line, induce either apoptosis or differentiation. Arrest at G11/S and G2/M is believed to be mediated by the concomitant upregulation of the CDK-inhibitor p21WAF1/Cip1 gene (Richon et al., Proc. Natl. Acad. Sci. USA 97: 10014-10019 (2000); Ju and Muller, Can. Res. 63: 2891-2897 (2003)). Apicidin is an example of an HDAC inhibitor ( Meinke and Liberator, Curr. Med. Chem. 8: 211-235 (2001), Colletti et al., Bioorg. Med. Chem. Letts. 11: 107-111 (2001), Colletti et al., Bioorg. Med. Chem. Letts. 11: 113-117 (2001), Colletti et al., Tetrahedron Letts. 41: 7825-7829 (2000), Meinke et al., Tetrahedron Letts. 41: 7831-7835 (2000), Colletti et al., Tetrahedron Letts. 41: 7837-7841 (2000), Meinke et al., J. Med. Chem. 43: 4919-4922 (2000), and Nare et al., Anal. Biochem. 267: 390-396 (1999)).

HDACs are recruited to target genes by protein complexes containing sequence-specific transcription factors and corepressors. At least 18 different human HDAC subtypes have been identified. These subtypes have been classified into three classes based on their homology to yeast proteins Rpd3 (Class I), Hda1 (Class II), and Sir2 (Class III). Aberrant patterns of histone acetylation have been linked to cancer and natural and synthetic HDAC inhibitors have been shown to have antiproliferative effects on tumor cells in culture and also to inhibit tumor growth in animal models. Thus, there has been increasing interest in HDAC inhibitors as novel antiproliferative or antitransformative agents which can be used to treat a variety of cancers. The use of HDAC inhibitors as novel drugs for treating various cancers has been reviewed by Johnstone in Nature Reviews/Drug Discovery 1: 287-299 (2002). In addition, U.S. Pat. No. 6,511,990 to Breslow et al. discloses compounds for treating cancers which are HDAC inhibitors; U.S. Pat. No. 6,541,661 to Delorme et al. discloses HDAC inhibitors which are useful for treating proliferative diseases; and, U.S. Pat. No. 6,638,530 to Ishibashi et al. discloses benzamide formulations which have HDAC inhibitor activity and which can be used as an anticancer agents. Reviews on the relationship between HDAC and cancer can be found in Vigushin and Coombes, Anti-Cancer Drugs 13: 1-13 (2002), Grozinger and Schreiber, Chem. Biol. 9: 3-16 (2002), Marks et al., Nature Reviews/Cancer 1: 194-202 (2001), Kramer et al., Trends Endocrin. Metabol. 12: 294-300 (2001), Cress and Seto, J. Cell. Physiol. 184: 1-16 (2000), and Archer and Hodin, Curr. Opin. Genet. Devel. 9: 171-174 (1999).

HDAC inhibitors have also been implicated for the treatment of other diseases or disorders as well. For example, U.S. Pat. No. 6,068,987 to Dulski et al. discloses an assay for identifying HDAC inhibitors for use as antiprotozoal agents. The assay entails contacting a protozoal cell with a test compound or natural product extract, isolating the histones, and determining the extent of acetylation of the histones. U.S. Pat. No. 6,387,673 to Evan et al. found that histone deacetylase associates with hormone receptor complexes and contributes to the repression of the hormone receptor. They further found that exposure of a repressed system to histone deacetylase inhibitors relieves this repression which suggests that histone deacetylase inhibitors can be useful for the activation of genes responsive to hormone receptors. U.S. Pat. No. 5,993, 845 to Geerts et al. discloses an HDAC inhibitor which is useful as an anti-fibrotic agent. U.S. Pat. No. 6,544,957 to Kern et al. discloses scriptaid which is an HDAC inhibitor that can be used to enhance transcription in cell-free and cell-based transactivation assays.

Although HDAC inhibitors appear to have great promise for treating a variety of diseases such as leukemias and solid tumors, protozoal diseases, and the like, therapeutic applications of currently available HDAC inhibitors are hampered by a number of limitations such as poor stability, PK profile, or potency. Therefore, there is a need for novel HDAC inhibitors which do not have the limitations of current HDAC inhibitors. Thus, there is a particular need for novel assays which can identify these HDAC inhibitors based upon biologically relevant targets.

### BRIEF SUMMARY OF THE INVENTION

The present invention provides a cell-based method for identifying novel histone deacetylase (HDAC) inhibitors. The method provides cells comprising a reporter gene operably linked to a transcription regulatory sequence, which includes nucleotide sequences responsive to a known HDAC inhibitor or subtype, stably integrated into the genome of the cells. A culture of the cells is incubated in a medium containing an analyte being tested for HDAC inhibitory activity and monitored over time for expression of the reporter gene. Analytes which have HDAC inhibitor activity induce expression of the reporter gene. In particular embodiments, the transcription regulatory region is a sequence of the p21^{WAF1/CIP1} transcription regulatory sequence which is responsive to a known HDAC inhibitor but not responsive to p53.

Therefore, in one embodiment, the present invention provides a method for identifying an analyte which is a histone deacetylase (HDAC) inhibitor, which comprises (a) providing cells which include a reporter gene encoding an enzyme operably linked to a transcription regulatory sequence which includes nucleotide sequences responsive to a known HDAC inhibitor or HDAC subtype stably integrated into the genome of the cells; (b) culturing the cells in a medium which includes the analyte and a substrate for the enzyme; and (c) measuring activity of the enzyme on the substrate wherein an increase in the activity of the enzyme on the substrate indicates that the analyte is an HDAC inhibitor.

In another embodiment, the present invention provides a method for treating a cancer in a patient, which comprises (a) providing one or more cultures of cells which include a reporter gene operably linked to a transcription regulatory sequence which includes nucleotide sequences responsive to a histone deacetylase (HDAC) inhibitor or HDAC subtype stably integrated into the genome of the cells; (b) culturing each of the one or more cultures of cells in a medium which contains an analyte; (c) identifying the analytes which stimulate expression of the reporter gene in the cells; and (d) administering one or more of the analytes identified in step (c) to stimulate expression of the reporter gene to the patient to treat the cancer. In a further aspect of the embodiment, the transcription regulatory sequence is a p21^{WAF1/CIP1} transcription regulatory sequence which includes nucleotide sequences responsive to a histone deacetylase (HDAC) inhibitor selected from the group consisting of Apicidin, Trichostatin A, sodium butyrate, SAHA, and MS27-275 and not nucleotide sequences responsive to p53.

In a further embodiment, the present invention provides a method for inducing differentiation or apoptosis of a proliferative cell, which comprises (a) providing one or more cultures of cells which include a reporter gene operably linked to a transcription regulatory sequence which includes nucleotide sequences responsive to a histone deacetylase (HDAC) inhibitor or HDAC subtype stably integrated into the genome of the cells; (b) culturing each of the one or more cultures of cells in a medium which contains an analyte; (c) identifying the analytes which stimulate expression of the reporter gene in the cells; and (d) administering one or more of the analytes identified in step (c) to stimulate expression of the reporter gene to the proliferative cell to induce the differentiation or apoptosis of the proliferative cell. In a further aspect of the embodiment, the transcription regulatory sequence is a p21^{WAF1/CIP1} transcription regulatory sequence which includes nucleotide sequences responsive to a histone deacetylase (HDAC) inhibitor selected from the group consisting of Apicidin, Trichostatin A, sodium butyrate, SAHA, and MS27-275 and not nucleotide sequences responsive to p53.

In a further still embodiment, the present invention provides a method for identifying an analyte which is a histone deacetylase (HDAC) inhibitor, which comprises (a) providing cells which include a reporter gene encoding an enzyme operably linked to a transcription regulatory sequence which includes nucleotide sequences responsive to a known HDAC inhibitor or HDAC subtype stably integrated into the genome of the cells; and (b) measuring expression of the reporter gene wherein an increase in expression of the reporter gene indicates that the analyte is an HDAC inhibitor.

In particular aspects of the above methods, the cells do not have a functional p53. In further aspects of the above methods, the cells are mammalian cells or human cells. In further aspects of the method, the cells which contain the reporter gene encoding an enzyme operably linked to the transcription regulatory sequence which includes nucleotide sequences responsive to a known HDAC inhibitor are selected from the group consisting of HeLa cells and MCF7 cells.

In further still aspects of the above methods, the known HDAC inhibitor is selected from the group consisting of Apicidin, Trichostatin A, sodium butyrate, SAHA, and MS27-275.

In particular aspects of the above methods, the transcription regulatory sequence includes a transcription regulatory sequence of p21WAF1/CIP1 which does not include a nucleotide sequences responsive to p53. In a preferred aspect, the p21WAF1/CIP1 transcription regulatory sequence includes from about nucleotide −183 to nucleotide +25 of the p21WAF1/CIP1 transcription regulatory sequence or the p21WAF1/CIP1 transcription regulatory sequence includes the nucleotide sequence set forth in SEQ ID NO: 1.

In further aspects of the above methods, the reporter gene encodes β-lactamase. In a further still aspect, the cells are ICLC PD02008 which are recombinant HeLa cells containing the reporter gene β-lactamase operably linked to a p21 minimal promoter which comprises the nucleotide sequence set forth in SEQ ID NO:1.

In further aspects of the above methods, the substrate for the β-lactamase includes a cephalosporin cleavage site which preferably is labeled with a donor:acceptor fluorophore pair which is capable of fluorescence resonance energy transfer. Examples of donor:acceptor fluorophore pairs include fluorescein as the donor fluorophore and coumarin as the acceptor fluorophore.

In a further still embodiment of the present invention, a method is provided for identifying analytes which are histone deacetylase (HDAC) inhibitors, which comprises (a) providing one or more cultures of cells which include a reporter gene encoding an enzyme operably linked to a transcription regulatory sequence responsive to an HDAC inhibitor or HDAC subtype stably integrated into the genome of the cells; (b) culturing each of the one or more cultures of recombinant cells in a medium which contains an analyte; and (c) identifying the analytes which stimulate expression of the reporter gene in the cells, wherein analytes which stimulate expression of the reporter gene are HDAC inhibitors. In a further aspect of this embodiment, the transcription regulatory sequence consists essentially of the transcription regulatory sequence from about nucleotide −183 to nucleotide +25 of the p21WAF1/CIP1 transcription regulatory sequence.

In further aspects of the above methods, the HDAC is a subtype selected from the group consisting of Class I HDACs, Class II HDACs, and Class III HDACs or further still, the known HDAC is a subtype selected from the group consisting of HDAC1, HDAC2, HDAC3, HDAC8, HDAC4, HDAC5, HDAC6, HDAC7, HDAC9, HDAC10, HDAC11, SIRT1, SIRT2, SIRT3, SIRT4, SIRT5, SIRT6, and SIRT7.

The present invention further provides a cell comprising a reporter gene encoding an enzyme operably linked to a transcription regulatory sequence which includes nucleotide sequences responsive to a known histone deacetylase (HDAC) inhibitor or HDAC subtype stably integrated into the genome of the cell and a cell comprising a reporter gene operably linked to a p21WAF1/CIP1 transcription regulatory sequence which includes nucleotide sequences responsive to a histone deacetylase (HDAC) inhibitor selected from the group consisting of Apicidin, Trichostatin A, sodium butyrate, SAHA, and MS27-275 and not nucleotide sequences responsive to p53 stably integrated into the genome of the cell.

In further aspects of the above cells, it is preferable that the cells do not have a functional p53. In further still aspects, the cells are mammalian cells or human cells. In further still aspects, the cells are selected from the group consisting of HeLa cells and MCF7 cells.

In a further aspect of the cell comprising a reporter gene operably linked to a p21WAF1/CIP1 transcription regulatory sequence, the p21WAF1/CIP1 transcription regulatory sequence does not include nucleotide sequences responsive to p53 and preferably includes the nucleotide sequence from about nucleotide −183 to nucleotide +25 of the p21WAF1/CIP1 transcription regulatory sequence or includes the nucleotide sequence set forth in SEQ ID NO:1. In a particular aspect, the cell comprising a reporter gene operably linked to a p21WAF1/CIP1 transcription regulatory sequence is ICLC PD02008.

In further aspects of the above cells, the reporter gene encodes β-lactamase.

In further aspects of the above cells, the HDAC is a subtype selected from the group consisting of Class I HDACs, Class II HDACs, and Class III HDACs or further still, the known HDAC is a subtype selected from the group consisting of HDAC1, HDAC2, HDAC3, HDAC8, HDAC4, HDAC5, HDAC6, HDAC7, HDAC9, HDAC10, HDAC11, SIRT1, SIRT2, SIRT3, SIRT4, SIRT5, SIRT6, and SIRT7.

The present invention further provides a plasmid comprising a gene encoding β-lactamase operably linked to a transcription regulatory sequence which includes nucleotide sequences responsive to a histone deacetylase (HDAC) inhibitor or HDAC subtype and a plasmid comprising a gene encoding β-lactamase operably linked to a p21WAF1/CIP1 transcription regulatory sequence which includes nucleotide sequences responsive to a histone deacetylase (HDAC) inhibitor selected from the group consisting of Apicidin, Trichostatin A, sodium butyrate, SAHA, and MS27-275 and not nucleotide sequences responsive to p53.

In a further aspect of the plasmid comprising a reporter gene operably linked to a p21WAF1/CIP1 transcription regulatory sequence, the p21WAF1/CIP1 transcription regulatory sequence includes the nucleotide sequence from about nucleotide −183 to nucleotide +25 of the p21WAF1/CIP1 transcription regulatory sequence or includes the nucleotide sequence set forth in SEQ ID NO: 1. In further aspects of the above plasmids, the reporter gene encodes β-lactamase.

In further aspects of the above plasmids, the HDAC is a subtype selected from the group consisting of Class I HDACs, Class II HDACs, and Class III HDACs or further still, the known HDAC is a subtype selected from the group consisting of HDAC1, HDAC2, HDAC3, HDAC8, HDAC4, HDAC5, HDAC6, HDAC7, HDAC9, HDAC10, HDAC11, SIRT1, SIRT2, SIRT3, SIRT4, SIRT5, SIRT6, and SIRT7.

The present invention further provides a kit, which comprises cells which include a reporter gene operably linked to a transcription regulatory sequence which includes nucleotide sequences responsive to a known histone deacetylase (HDAC) inhibitor or HDAC subtype stably integrated into the genome of the cells. In a further aspect of the kit, the kit further comprises a substrate for the reporter gene. In particular embodiments, the reporter gene is operably linked to a p21WAF1/CIP1 transcription regulatory sequence which does not nucleotide sequences responsive to p53. Preferably, the p21WAF1/CIP1 transcription regulatory sequence includes the nucleotide sequence from about nucleotide −183 to nucleotide +25 of the p21WAF1/CIP1 transcription regulatory sequence or includes the nucleotide sequence set forth in SEQ ID NO: 1. In a further aspect, the cell comprising a reporter gene operably linked to a p21WAF1/CIP1 transcription regulatory sequence is PD02008. In further still aspects, the reporter gene encodes β-lactamase and the substrate for the β-lactamase includes a cephalosporin cleavage site which preferably is labeled with a donor:acceptor fluorophore pair which is capable of fluorescence resonance energy transfer. Examples of donor:acceptor fluorophore pairs include fluorescein as the donor fluorophore and coumarin as the acceptor fluorophore.

In further aspects of the above kit, the HDAC is a subtype selected from the group consisting of Class I HDACs, Class II HDACs, and Class III HDACs or further still, the known HDAC is a subtype selected from the group consisting of HDAC1, HDAC2, HDAC3, HDAC8, HDAC4, HDAC5, HDAC6, HDAC7, HDAC9, HDAC10, HDAC11, SIRT1, SIRT2, SIRT3, SIRT4, SIRT5, SIRT6, and SIRT7.

The present invention further provides a method for identifying an analyte which is a histone deacetylase (HDAC) inhibitor, which comprises (a) providing cells which include a reporter gene encoding an enzyme operably linked to a transcription regulatory sequence which includes nucleotide sequences responsive to a known HDAC stably integrated into the genome of the cells; (b) culturing the cells in a medium which includes the analyte and a substrate for the enzyme; and (c) measuring activity of the enzyme on the substrate wherein an increase in the activity of the enzyme on the substrate indicates that the analyte is an HDAC inhibitor.

The present invention further provides a method for identifying an analyte which is a histone deacetylase (HDAC) inhibitor, which comprises (a) providing a transcription regulatory sequence of a gene which is responsive to a known HDAC; (b) constructing a gene expression cassette comprising a reporter gene encoding an enzyme operably linked to the transcription regulatory sequence of the gene in a plasmid; (c) transfecting a cell with the gene expression cassette in a plasmid to produce a cell which includes the gene expression cassette stably integrated into the genome of the cell; (d) providing a multiplicity of the cell in a medium which includes the analyte and a substrate for the enzyme; and (e) measuring activity of the enzyme on the substrate wherein an increase in the activity of the enzyme on the substrate indicates that the analyte is an HDAC inhibitor.

The present invention further provides a cell comprising a reporter gene encoding an enzyme operably linked to a transcription regulatory sequence which includes nucleotide sequences responsive to a known histone deacetylase (HDAC) stably integrated into the genome of the cell.

In further aspects of the above, the cells are mammalian cells or human cells. In further still aspects, the cells are selected from the group consisting of HeLa cells and MCF7 cells.

In further aspects of the above, the known HDAC is a subtype selected from the group consisting of Class I HDACs, Class II HDACs, and Class III HDACs or further still, the known HDAC is a subtype selected from the group consisting of HDAC1, HDAC2, HDAC3, HDAC8, HDAC4, HDAC5, HDAC6, HDAC7, HDAC9, HDAC10, HDAC11, SIRT1, SIRT2, SIRT3, SIRT4, SIRT5, SIRT6, and SIRT7.

In further still aspects of the above, the known HDAC inhibitor is selected from the group consisting of Apicidin, Trichostatin A, sodium butyrate, SAHA, and MS27-275.

In further still aspects of the above, the reporter gene encodes β-lactamase and further still, the substrate for the β-lactamase includes a cephalosporin cleavage site. In any of the above, the substrate is labeled with a donor:acceptor fluorophore pair which is capable of fluorescence resonance energy transfer.

In further still aspects of the above, the transcription regulatory sequence of the gene is provided by (i) transfecting cells with a small interfering RNA (siRNA) that selectively inhibits the expression of the known HDAC; (ii) measuring expression of mRNAs produced by the cells transfected with the siRNA to the expression of the mRNAs produced in cells not transfected with the siRNA, wherein mRNAs which have increased expression in the cells transfected with the siRNA are encoded by genes which have a transcription regulatory sequence responsive to the known HDAC; (iii) identifying at least one of the genes encoding an mRNA of the mRNAs which have increased expression in the cells transfected with the siRNA; and (iv) isolating the transcription regulatory sequence to the gene to provide the transcription regulatory sequence, or the transcription regulatory sequence of the gene is provided by (i) transfecting cells with an antisense oligonucleotide (ASO) that selectively inhibits the expression of the known HDAC; (ii) measuring expression of mRNAs produced by the cells transfected with the ASO to the expression of the mRNAs produced in cells not transfected with the ASO, wherein mRNAs which have increased expression in the cells transfected with the ASO are encoded by genes which have a transcription regulatory sequence responsive to the known HDAC; (iii) identifying at least one of the genes encoding an mRNA of the mRNAs which have increased expression in the cells transfected with the ASO; and (iv) isolating the transcription regulatory sequence to the gene to provide the transcription regulatory sequence.

### BRIEF DESCRIPTION OF THE DRAWINGS

FIG. 1 shows the nucleotide sequence of the minimal p21 promoter (SEQ ID NO:1).

FIG. 2A shows a map of the plasmid p21m-BLA which contains the Bla gene operably linked to the minimal p21 promoter.

FIGS. 2B to 2D shows the nucleotide sequence of plasmid p21m-BLA (SEQ ID NO:4). The BglII and KpnI restriction endonuclease cleavage sites flanking the p21 minimal promoter are under lined. The p21 minimal promoter is in lower case and the Bla gene is in italics.

FIG. 3 is an immunoblot showing HeLa cells have the best ratio between 1 μM Apicidin induced p21 expression to background p21 protein expression when compared to p21 expression in HCT116, MCF7, and Hep3b cells. The immunoblot was probed with a p21-specific antibody.

FIG. 4A shows the green fluorescence of clone 1.17 cells incubated in the presence of DMSO and CFF2 substrate. DMSO does not induce expression of the Bla gene. Therefore, no β-lactamase is produced and the CFF2 substrate remains intact. Because the fluorescein remains in close proximity to the coumarin, FRET takes place between the fluorescein and the coumarin which is detectable as a green fluorescence.

FIG. 4B shows the blue fluorescence of clone 1.17 cells incubated in the presence of 1 μM Apicidin and CFF2 substrate. The Apicidin induces expression of the Bla gene. Therefore, β-lactamase is produced which then cleaves the CFF2 substrate which separates the fluorescein from the coumarin. The blue fluorescence is produced by the fluorescein.

FIG. 5 is a photograph of the products of a PCR reaction of various clones containing p21m-BLA using primers for amplifying the p21m-BLA electrophoresed on an Agarose gel and stained with ethidium bromide. Arrow points the DNA band corresponding to p21m-BLA. −/− means no β-lactamase activity in presence of DMSO or HDAC inhibitor, +/+ means β-lactamase activity in presence of both DMSO and HDAC inhibitor; and −/+ means β-lactamase only in presence of HDAC inhibitor.

### DETAILED DESCRIPTION OF THE INVENTION

The present invention provides a novel cell-based method for identifying histone deacetylase (HDAC) inhibitors. The method is particularly useful for high throughput screening assays to identify HDAC inhibitors. Because HDAC inhibitors can have an antiproliferative effect on tumor cells in culture, analytes identified using the method herein are useful for treating a variety of cancers and leukemias in humans and animals. Furthermore, particular analytes identified by the method herein can also have antiprotozoal, antifungal, and antiviral applications as well. An HDAC inhibitor can either inhibit expression of the HDAC or inhibit HDAC deacetylase activity.

The elements of the method comprise culturing a cell line comprising recombinant cells which have stably integrated into the genome of the recombinant cells a gene expression cassette comprising a reporter gene operably linked to a transcription regulatory sequence (or promoter) of a transcription unit (or gene) which is responsive to, upregulated, or activated when a known HDAC inhibitor is administered to the recombinant cell in a medium containing an analyte being tested for its potential to inhibit HDAC. An analyte which has HDAC inhibitory activity will induce or activate expression of the reporter gene whereas an analyte without HDAC inhibitory activity will not induce or activate expression of the reporter gene. Expression of the reporter gene is determined by measuring the amount of reporter gene product made or the activity of the reporter gene on a particular substrate over time. In preferred aspects of the method, a negative control is provided which comprises culturing the recombinant cells in a medium without the analyte and a positive control is provided which comprises culturing the recombinant cells in a medium with a known HDAC inhibitor which is capable of activating expression of the reporter gene.

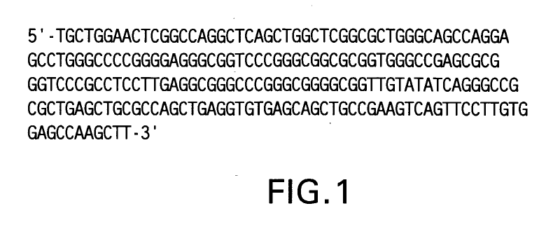

Because almost all known HDAC inhibitors activate transcription of cyclin-dependent kinase (CDK) inhibitor WAF1 (also known as CIP1, p21, or p21^{WAF1/CIP1}), in particular aspects of the present invention, it is currently preferable that the nucleotide sequence encoding the reporter gene be operably linked to the 5′ transcription regulatory nucleotide sequence (or promoter) of the p21^{WAF1/CIP1} transcription unit. The 5′ transcription regulatory sequence of the p21^{WAF1/CIP1} transcription unit includes about 5,000 nucleotides which includes several p53 dependent regulatory sequences and several p53 independent regulatory sequences. The nucleotide sequence of the about 5,000 nucleotide 5′ transcription regulatory sequence is available from GenBank as Accession Nos. Z85996, AF497972, and U24170 and has been described in U.S. Pat. Nos. 5,807,692 and 5,871,968, both to Kinzier et al. In the present invention, it is preferable that the 5′ transcription regulatory sequence of the p21^{WAF1/CIP1} transcription unit exclude the p53 regulatory sequences and the upstream p53-independent regulatory sequences. This prevents expression of the reporter gene by p53 or other transcription activators which require one or more of the upstream p53-independent regulatory sequences. Therefore, in a preferred embodiment, the 5′ transcription regulatory sequence comprises the minimal nucleotide sequences needed for activation of transcription in response to an HDAC inhibitor. The minimal nucleotide sequence has been determined to include the nucleotides from −183 to about +25 bp of the p21^{WAF1/CIP1} promoter (hereinafter, the minimal nucleotide sequence of p21^{WAF1/CIP1} needed for activation by an HDAC inhibitor such as Apicidin, Trichostatin A, sodium butyrate, SAHA, or MS27-275 is referred to as the “p21 minimal promoter”). The nucleotide sequence of the minimal p21 promoter is shown in FIG. 1 (SEQ ID NO:1). Because there are variants of p21^{WAF1/CIP1}, the p21 minimal promoter can include the corresponding region in a p21^{WAF1/CIP1} variant.

Expression vectors consisting of various sequences of the 5′ regulatory sequence of p21^{WAF1/CIP1} operably linked to a reporter gene have been described. Egawa et al. (Biol. Pharm. Bull. 21: 899-904 (1998)) describe stable transformants of Saos-2 and TMK-1 cells (p53 defective and mutant form of p53, respectively) containing an integrated copies of either the full-length or a 210 bp PstI/HindIII fragment of p21^{WAF1/CIP1} 5′ regulatory sequence operably linked to a luciferase gene. The cells were used to screen Streptomyces culture fluids for transcriptional activators. Actinomycin and Trichostatin A were identified as activators. Perez et al. (Proc. Am. Assoc. Cancer Res. 43: 740, #3671 (2002)) describe a p21 promoter activation assay consisting of H1299 human cells (p53 deficient) transiently transfected with the p21^{WAF1/CIP1} promoter directing luciferase expression. The assay was used to identify an HDAC inhibitor. Furumai et al. (Cancer Res. 62: 4916-4921 (2002)) describe a stable transformant of My1Lu cells (mink lung epithelial cell line) containing integrated copies of a 2.4 kb regulatory sequence of operably linked to the luciferase gene. The cells were used to compare the HDAC inhibitory activity in vivo of FK228 and its reduced form redFK. U.S. Pat. Nos. 5,807,692 and 5,871,968, both to Kinzler et al. disclose plasmid vectors comprising the regulatory region of p21^{WAF1} operably linked to a gene. In one aspect, the plasmid vectors can be used to express a foreign gene in cells in which p21^{WAF1} is normally expressed. In another aspect, the foreign gene is a reporter gene which enables the user to assay for compounds which increase or reduce expression of p21^{WAF1}. In further aspects, the reporter gene is operably linked to upstream p53 recognition sequences or a p53-independent sequence. WO2003020930 to Roninson et al. discloses assays for identifying inhibitors of p21 expression comprising cells transfected with a reporter gene operably linked to a p21 promoter and WO995020280 to El Deiry et al. discloses assays for identifying inhibitors of BRCA1 and p53 complex formation comprising cells transfected with a reporter gene operably linked to a p21 promoter. Activation of p21 transcription by various compounds, including HDAC inhibitors, using different sized regulatory sequences of the p21 promoter operably linked to the luciferase gene has been studied in Han et al., J. Biol. Chem. 276: 42084-42090 (2001); Huang et al., Oncogene, 19: 5712-5719 (2000); Sambucetti et al., J. Biol. Chem. 274: 34940-34947 (1999); Xiao et al., J. Cell. Biochem. 73: 291-302 (1999); Sowa et al., Cancer Res. 59: 4266-4270 (1999); Nakano et al., J. Biol. Chem. 272: 22199-22206 (1997); Sowa et al., Biochem. Biophys. Res. Comm. 241: 142-150 (1997); Archer et al., Proc. Natl. Acad. Sci. USA 95: 6791-6796 (1998); and Rong and Muller, Cancer Res. 63: 2891-2897 (2003).

While in currently preferred aspects of the present invention the gene expression cassette comprises the reporter gene operably linked to the minimal p21 promoter, the 5′ regulatory sequence or minimal sequence therein from another gene or transcription unit, which is responsive to HDAC or an HDAC inhibitor, can be used in place of the 5′ regulatory sequence or minimal regulatory sequence of p21^{WAF1/CIP1}. Examples of other genes which are induced or activated by various families of HDAC inhibitors include, but are not limited to, CDKN1A (CDK inhibitor 1A), CDKN1B (CDK inhibitor 1B), GATA2 (GATA-binding protein), PKCD (protein kinase C-δ), MHC1 (major histocompatibility complex 1), MHC2 (major histocompatibility complex 2), BAK (BCL2 antagonist/killer protein), BAX (B-cell associated X protein), IL8 (interleukin 8), RARβ (retinoic acid receptor β), TG1 (transglutaminase type 1), cyclin E, CPA3 (carboxypeptidase A3), CD86, ICAM1 (intercellular adhesion molecule 1), β-catenin, HSP86 (heat shock protein 86), IGFBP3 (insulin-like growth factor binding protein 3), DHFR (dihydrofolate reductase), TGFB1 (transforming growth factor-β1), ER (estrogen receptor), IFNγ (interferon-γ), INFβ (interferon-β), TP53 (tumor protein p53 (Li-Fraumeni syndrome)), VHL (Von Hippel-Lindau syndrome protein), P107, MLH1, TIMP3 (tissue inhibitor of metalloproteinase 3), CD95, CD95L, gelsolin, PAI2 (plasminogen activator inhibitor type 2), MAGE3 (melanoma antigen family A3), NY-ESO1 (cancer/testis antigen), and TGFBR2 (transforming growth factor-β receptor 2) (See Johnstone, Nature Rev. 1: 287-299 (2002). Preferably, the regulatory sequence for any one of the above genes is modified to remove p53 regulatory sequences and regulatory sequences non-responsive to a known HDAC inhibitor. This avoids expression due to p53. In further aspects, the 5′ regulatory sequence can comprise one or more HDAC inhibitor responsive sequences from any combination of genes or transcription units responsive to a known HDAC inhibitor or HDAC subtype. Thus, the method includes using a transcription regulatory sequence responsive to a particular HDAC such as an HDAC subtype selected from the group consisting of Class I HDACs, Class II HDACs, and Class III HDACs or further still, an HDAC subtype selected from the group consisting of HDAC1, HDAC2, HDAC3, HDAC8, HDAC4, HDAC5, HDAC6, HDAC7, HDAC9, HDAC10, HDAC11, SIRT1, SIRT2, SIRT3, SIRT4, SIRT5, SIRT6, and SIRT7. In general, these responsive sequences allow the HDAC to downregulate expression of the transcription unit or gene. When expression of HDAC is inhibited, expression of the gene or transcription unit is upregulated. Thus, the method enables identification of HDAC inhibitors that inhibit the enzymatic activity of HDAC, interfere with binding of the HDAC to a transcription regulatory sequence, inhibit expression of HDAC or activate expression of a protein which interferes with the binding of the HDAC to a transcription regulatory sequence.

Current known HDAC inhibitors include those in the following structural classes: short-chain fatty acids (SCFA), hydroxamic acid (HA), cyclic tetrapeptides (CT), HA-CT hybrids, tetrapeptides, epoxides, ortho amino bezamides, and electrophilic carbonyls. Various HDAC inhibitors activate particular combinations of the above genes and have particular in vitro effects on cells (See Johnstone (2002) above). For example, butyrates are HDAC inhibitors of the SCFA structural class which induce apoptosis, differentiation, or cell-cycle arrest whereas Apicidin is an HDAC inhibitor of the CT structural class which induces apoptosis and cell-cycle arrest and valproic acid is an HDAC of the SCFA structural class which induces apoptosis or differentiation. In addition, particular HDAC inhibitors appear to affect transcription of particular families of genes. For example, Apicidin activates gelsolin whereas valproic acid activates β-catenin. Therefore, the 5′ transcription regulatory sequences from particular genes activated by particular HDAC inhibitors (such as any one of the genes enumerated above) can be operably linked to a reporter gene for use in the method herein to identify HDAC inhibitors with particular effects.

The reporter gene operably linked to the 5′ regulatory sequence of an HDAC inhibitor-inducible transcription unit or the minimal p21 promoter (SEQ ID NO:1) can be any reporter gene which produces and assayable product. Examples of reporter genes include the β-lactamase (Bla) gene, β-galactosidase (lacZ) gene, green fluorescence protein (GFP) gene, chloramphenicol acetyl transferase (CAT) gene, uroporphyrinogen III methyltransferase (cobA) gene, secreted alkaline phosphatase (SEAP) gene, and β-glucoronidase (Gluc) gene. In one aspect of the method, expression of the reporter gene operably linked to a regulatory sequence responsive to or activated by an HDAC inhibitor is determined by measuring the activity of the reporter gene product on a substrate for the reporter gene. Activity can be cleavage of a substrate, phosphorylation of a substrate, binding of a ligand or antibody to a substrate, enzyme activity, or the like. The detection of the reporter gene product or its activity in the presence of a test analyte indicates that the analyte has HDAC inhibitory activity. In currently preferred aspects of the present invention, the reporter gene encodes an enzyme which cleaves a substrate which is labeled, preferably a substrate which is labeled with a donor:acceptor fluorophore pair which exhibits fluorescence resonance energy transfer (FRET) and which produces a measurable shift or change in fluorescence upon cleavage by the reporter enzyme or a substrate which is labeled with a fluorophore which produces a detectable fluorescence upon cleavage by the reporter enzyme.

FRET is currently preferred for measuring cleavage of a substrate by the reporter gene product or in various modifications of a substrate by the reporter gene product. Thus, the substrate for the reporter gene product preferably comprises a donor:acceptor pair of fluorophores which are capable of FRET. For example, in the case of β-lactamase, the labeled substrate can comprise a cephalosporin cleavage site such as CCF2 which is labeled at one end with a donor fluor such as 6-fluoro-hydroxycoumarin and at the other end with an acceptor fluor such as fluorescein (Zlokarnik et al., Science 279: 84-88 (1988); Xing et al., J. Recept. Signal Transduct. Res. 20: 189-210 (2000); Galarneau et al., Nature Biotech. 20: 619-622 (2002); above patents to Tsien et al.). Other donor:acceptor fluorophore pairs can also be used. In the case of the labeled CFF2 substrate, the labeled CFF2 substrate is delivered to cells in a form (-AM ester) which leaves it uncharged so it can easily cross the plasma membrane of many cell types. Upon entering the cell, the AM ester is cleaved by β-lactamase, which traps the cleaved products in the cell. In cells that take up CCF2 substrate labeled with coumarin and fluorescein, for example, but which do not make the β-lactamase, the labeled CFF2 remains intact and light at 405 nm excites the coumarin to a higher excitation level. Energy from its decay back to the ground state is transferred to the fluorescein which produces a green fluorescence at 525 nm. In cells that do make β-lactamase, the labeled CFF2 is cleaved and there is no FRET to fluorescein. Light at 405 nm excites the coumarin which produces a blue fluorescence at 450 nm. Because β-lactamase is responsible for the cleavage of the antibiotic penicillin, there are also suicide inhibitors to the β-lactamase. Suicide inhibitors allow the user to kill off any active β-lactamase in the cells, at the moment of their choosing, and then add the analyte and substrate, to observe de novo expression of the β-lactamase. CCF2 substrate labeled with 6-fluoro-hydroxycoumarin and fluorescein is available from Aurora Biosciences and Invitrogen, Carlsbad, Calif. Other examples of labeled substrates for β-lactamase are disclosed in U.S. Pat. Nos. 6,472,205, 6,291,162, 5,955,604, and 5,741,657, and WO9630540, all to Tsien et al.

In a currently preferred aspect, the reporter gene encodes the enzyme β-lactamase and the method preferably includes a labeled substrate for the β-lactamase which comprises a donor:acceptor fluorophore pair which is capable of FRET and which produces a measurable shift or change in fluorescence upon cleavage by the β-lactamase. Such a labeled substrate can be a molecule comprising a cephalosporin cleavage site such as CCF2 (Zlokarnik et al., Science 279: 84-88 (1988)). Other substrates for the β-lactamase include those disclosed in U.S. Pat. Nos. 6,472,205, 6,291,162, 5,955,604, and 5,741,657, and WO9630540, all to Tsien et al. It is further currently preferable that the β-lactamase gene be optimized for expression in eukaryote cells and retention in eukaryote cells, particularly for expression and retention in mammalian cells. For example, an optimized β-lactamase gene encodes a cytosolic form of β-lactamase which lacks the signal secretory sequence. Thus, the cytosolic β-lactamase is not secreted, it is retained in the cell. An example of a gene encoding a cytosolic β-lactamase is disclosed in U.S. Pat. Nos. 6,291,162 and 6,472,205, both to Tsien et al. Transcription activation of the β-lactamase gene is determined by measuring the amount of a labeled substrate for the β-lactamase which is cleaved in the presence of the analyte. Cleavage of the substrate in the presence of the analyte indicates that the analyte activated or induced expression of the β-lactamase gene and is, therefore, an HDAC inhibitor. In this aspect of the method, it is currently preferable that the recombinant cells comprise a reporter gene such as the β-lactamase gene operably linked to the 5′ transcription regulatory sequence of an HDAC inhibitor activatable transcription unit such as p21^{WAF1/CIP1} (preferably, the minimal p21 promoter described above) stably integrated into the genome of cells which, preferably, do not produce a functional p53. The cells are cultured in the presence of a test analyte for a time sufficient for the analyte to induce expression of the reporter gene (if the analyte is an HDAC inhibitor). Then, labeled substrate for the reporter gene product is added to the culture and activity of the reporter gene product is measured by detecting cleavage of the labeled substrate. In some aspects the analyte and labeled substrate are added to the cells at the same time. In other aspects, the analyte is added before the substrate is added to the cells and in other aspects, the substrate is added to the cells before the analyte. β-lactamase-based reporter systems and substrates are commercially available from Aurora Biosciences Corp., San Diego, Calif. and have been disclosed in U.S. Pat. Nos. 6,472,205, 6,291,162, 5,955,604, and 5,741,657, and WO9630540, all to Tsien et al.

While it is currently preferred that the reporter gene be β-lactamase, in another aspects, the reporter gene can be the green fluorescence protein (GFP) gene, uroporphyrinogen III methyltransferase (cobA) gene, β-galactosidase (LacZ) gene, β-glucoronidase (Gluc) gene, secreted alkaline phosphatase (SEAP) gene, chloramphenicol acetyl transferase (CAT) gene, or the like. In a manner similar to the β-lactamase gene, the above reporter genes are operably linked to a regulatory sequence responsive to or activated by an HDAC inhibitor, for example, the minimal p21 promoter, and expression is determined by measuring activity of the reporter gene product or the amount of reporter gene product produced. Detectable expression of the reporter gene in the presence of a test analyte over negative controls indicate that the analyte has HDAC inhibitory activity. Therefore, in this aspect of the method, the recombinant cells can comprise a reporter gene, for example, green fluorescence protein (GFP) gene, operably linked to the 5′ transcription regulatory sequence of an HDAC inhibitor responsive or activatable transcription unit (gene expression cassette) such as p21^{WAF1/CIP1} (preferably the p21 minimal promoter described above) stably integrated into the genome of cells which preferably do not produce a functional p53. The cells are cultured in the presence of a test analyte for a time sufficient for the analyte to induce expression of the reporter gene (if the analyte is an HDAC inhibitor). In the above example, expression of the green fluorescence protein indicates that the analyte is an HDAC inhibitor. Alternatively, the reporter gene can comprise β-galactosidase gene and expression is measured by detecting fluorescence produced by cleavage of the fluorescein di-β-galactopyranoside by the β-galactosidase or cleavage of a FRET labeled substrate.

Currently, an enzyme reporter gene cassette which allows FRET-based detection is preferred. It offers a flexible and sensitive assay development and screening platform than many other reporter systems where the amount reporter product is measured or where a chromophore substrate is used. In FRET-based detection systems, by obtaining a ratio of two emission intensities, the assay read-out is minimally affected by variation in cell size, cell number, probe concentration, and light intensity. Furthermore, the broad applicability and suitability for both developing and running ultra-high throughput screening (UHTPS) miniaturized assays gives it distinctive advantages over other approaches. Furthermore, the ability to confirm results by visual inspection allows for quick and simple diagnosing of assay performance without specialized instrumentation. By comparison, long read out times required for luciferase reduces its practical utility for UHTPS. GFP is a fluorescence-based reporter of single cell gene expression whose assay is less invasive than that of β-lactamase. However, GFP is non enzymatic and exhibits a delay before fluorescence development. This results in reduced sensitivity compared to β-lactamase. Typically, β-lactamase activity has a detection limit of around 50 molecules/cell, whereas GFP has a detection limit (that is set by the auto-fluorescent background of the cell) of around 1×10⁵ molecules/cell. In light of the above, the method of the present invention which uses the β-lactamase reporter gene cassette operably linked to the HDAC inhibitor activatable or responsive regulatory sequence is suitable for miniaturization given its high signal to noise ratio and high sensitivity and can be used for UHTPS in an automated fashion. Other transcriptional assays which are based on other enzyme reporter genes such as alkaline phosphatase, chloramphenicol acetyl transferase, or β-galactosidase either are not easily miniaturizable, are not simple to automate, or do not have the same sensitivity.

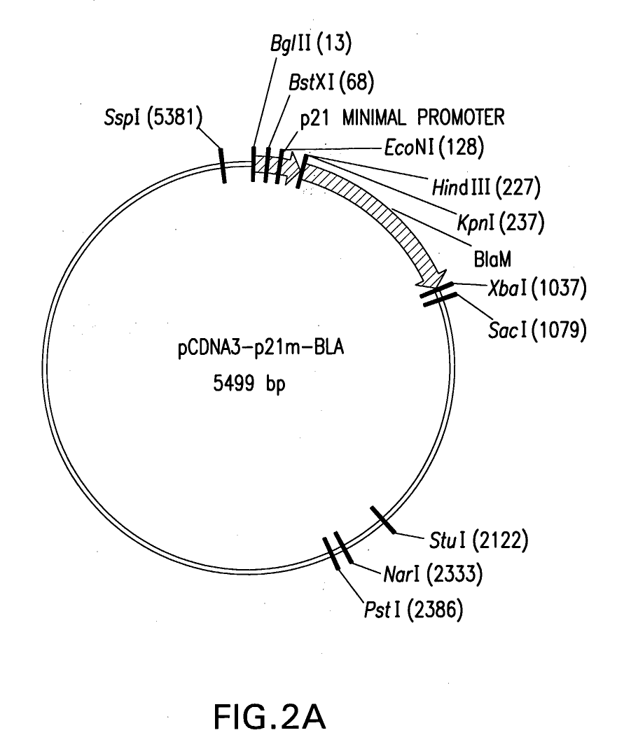

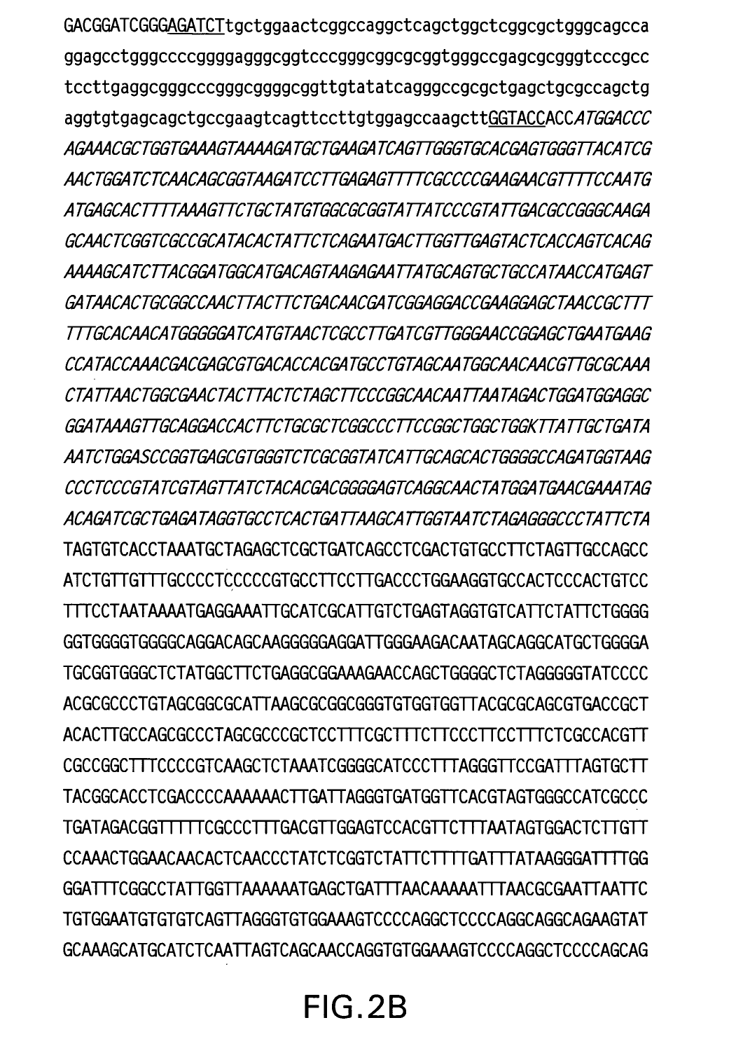

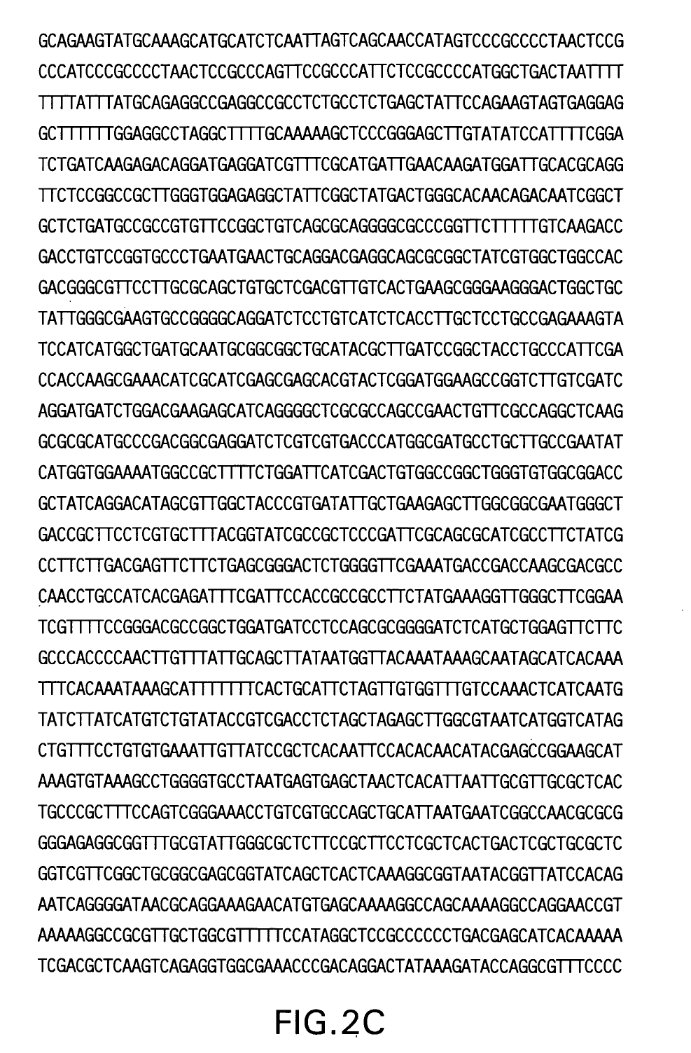

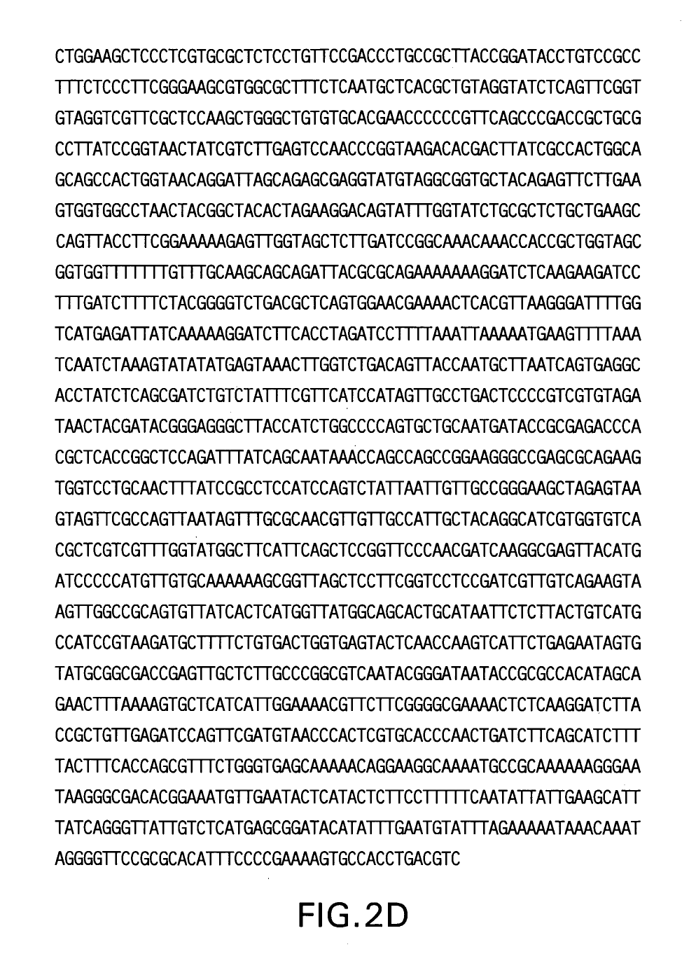

In a currently preferred aspect of the present invention, the method provides a recombinant cell comprising a reporter gene operably linked to an HDAC inhibitor activatable regulatory sequence (gene expression cassette) stably integrated into the genome of the cell. Preferably, the recombinant cell is a mammalian or human cell. Examples of cells which are useful include, but are not limited to, HeLa (American Type Culture Collection (ATCC) CCL-2), Hep3b (ATCC HB-8064), WiDr (ATCC CCL-218), HCT116 (ATCC CCL-247), MCF-7 (ATCC HTB-22), and 293 (ATCC CRL-1573). Preferably, the recombinant cells are human cells such as HeLa or MCF7 cells, particularly when the promoter is the p21 promoter or a sequence thereof such as the p21 minimal promoter. In a preferred aspect of the embodiment, the reporter gene encodes β-lactamase operably linked to a transcription regulatory sequence operably linked to a gene whose expression is regulated by HDAC. In a further aspect, the reporter gene is operably linked to a regulatory sequence comprising nucleotides −185 to +25 flanking the p21 transcription start site (minimal p21 promoter). For example, the reporter gene expression cassette can comprise the p21m-BLA gene cassette, which is illustrated in FIG. 2A and which includes the nucleic acid sequence shown in FIG. 2B inserted into plasmid pcDNA3. A recombinant HeLa cell line designated clone 1.17, which comprises a reporter gene encoding β-lactamase operably linked to the minimal p21 promoter inserted into pcDNA3 was deposited at the Centro di Biotecnologie Avanzate (CBA), Interlab Cell Line Collection (ICLC), Servizio Biotecnologie, L.go Rosanna Benzi, 10, 16132 Genova, Italia on Nov. 20, 2002, under the terms of the Budapest Treaty as deposit ICLC PD02008. The above recombinant HeLa cell line has been constructed as set forth in Example 2 and can be used in the method of the present invention. The above recombinant HeLa cell line is an adherent cell line which renders the cells suitable for culturing in tissue culture dishes and microtiter plates and for many high throughput screening formats.

In general, the method of the present invention involves incubating recombinant cells comprising a reporter gene operably linked to an HDAC inhibitor inducible promoter in a culture medium and under conditions suitable for growing the cells. Preferably, the reporter gene operably linked to the HDAC inhibitor inducible promoter is stably integrated into the genome of the recombinant cells. For example, the above recombinant cells can be recombinant HeLa cells which can be grown in Dulbecco's modified Eagles medium (DMEM) supplemented with about 5% fetal bovine serum at 37° C. in the presence of about 5% CO₂. A test analyte being tested for HDAC inhibitor activity is then added to the medium and the cells incubated for a time sufficient for expression of the reporter gene. The time sufficient for expression of the reporter gene can be determined for any particular HDAC inhibitor inducible promoter by incubating the cells in the presence of a known HDAC inhibitor and then measuring expression of the reporter gene at various times afterwards. In general, incubating the cells for a time greater than about 5 hours and preferably for a time at least about 13 hours in the presence of the analyte which is an HDAC inhibitor is sufficient to produce detectable reporter gene product. After sufficient time has been allowed to elapse for expression of the reporter gene, expression of the reporter gene is detected. Detection of expression can be indirect which is measured by adding a substrate for the reporter gene product to the culture and detecting modification of the substrate or by directly detecting the reporter gene product.

Indirect detection of expression of the reporter gene, which is performed by measuring activity of the reporter gene product on a substrate, can include any of the following. For the reporter enzyme β-lactamase, a substrate comprising a cephalosporin cleavage site labeled with a donor:acceptor pair of fluorophores capable of FRET in the intact substrate and which are situated on opposite sides of the cleavage site is provided. Cleavage at the cleavage site by the reporter enzyme β-lactamase causes a measurable change or shift in fluorescence. Alternatively, a substrate is provided which comprises a cephalosporin cleavage site labeled with a fluorophore which is not detectable when the substrate is intact but which is detectable when the substrate is cleaved by β-lactamase. For the reporter enzyme β-galactosidase, a substrate which comprises a β-galactosidase cleavage site labeled with a donor:acceptor pair of fluorophores capable of FRET in the intact substrate and which are situated on opposite sides of the cleavage site is provided. Cleavage at the cleavage site by the reporter enzyme β-galactosidase causes a measurable change or shift in fluorescence. Alternatively, a fluorogenic substrate is provided which comprises a β-galactosidase cleavage site labeled with fluorophore which is not detectable when the substrate is intact substrate but which is detectable when the substrate is cleaved (e.g., fluorescein di-β-galactopyranoside which releases fluorescein when cleaved). For a reporter enzyme which is a kinase, a substrate is provided which is phosphorylated by the kinase. The phosphorylated substrate is detected. For a reporter enzyme which is a phosphatase such as alkaline phosphatase, a fluorogenic substrate is provided which produces fluorescence when dephosphorylated by the phosphatase. For a reporter enzyme which is a peptidase or protease, a fluorogenic peptide which comprises a cleavage site specific for a particular peptidase or protease labeled with a donor:acceptor pair of fluorophores capable of FRET in the intact substrate and which are situated on opposite sides of the cleavage site is provided. Cleavage at the cleavage site by the peptidase or protease causes a measurable change or shift in fluorescence. Other reporter enzyme and substrate combinations not listed above can be used to similar effect.

Direct detection of reporter gene expression is performed by measuring the amount of reporter gene product made. For example, the reporter gene product can be detected using labeled antibodies specific for the reporter gene product or a labeled ligand which binds the reporter gene product, or directly such as when the reporter gene product is luciferase, green fluorescence protein, or the like.

Whether the detection is direct or indirect, the method can be performed in any suitable format such as in a 96-well microplate format.

In particular aspects of the method, it is desirable further include one or more positive controls. Known HDAC inhibitors which are useful in positive controls include, but are not limited to, compounds of the short chain fatty acid (SCFA) structural family such as butyrates and valproic acid; compounds of the hydroxamic acid (HA) structural family such as Trichostatin A (TSA), suberoylanilide hydroxamic acid (SAHA), m-carboxy cinnamic acid bishydroxamic acid (CBHA), and oxamflatin; compounds of the cyclic tetrapeptide (CT) structural family such as trapoxin A and Apicidin; CHAP an HA-CT hybrid; compounds of the tetrapeptide structural family such as FR901228 (depsipeptide); compounds of the epoxide structural family such as depudecin; and, compounds of the benzamidine structural family such as MS-27-275. The known HDAC inhibitors are useful as controls in the method of the present invention for identifying transcription regulatory sequences which are responsive to HDAC inhibitors.

In particular aspects of performing the method, aliquots of the cells are provided. Each aliquot is deposited in the well of a microplate. Serial dilutions of a test analyte being tested for HDAC inhibitor activity are made and each dilution is added to a separate well of the microtiter plate containing the cells. Optionally, the method can include serial dilutions of a known HDAC inhibitor as a positive control and further can include negative controls. In other aspects of performing the method, aliquots of the cells are provided. Each aliquot is deposited in the well of a microplate. A plurality of test analytes being tested for HDAC inhibitor activity are each added to a separate well of the microtiter plate containing the cells. Optionally, the method can include serial dilutions of a known HDAC inhibitor as a positive control and further can include negative controls. In further aspects, the serial dilutions of the plurality of analytes is made and each dilution is added to a separate well of the microtiter plate containing the cells.

In a currently preferred aspect, the method uses recombinant cells comprising a reporter gene encoding β-lactamase operably linked to the minimal p21 promoter, for example, the HeLa cells disclosed herein and deposited under the terms of the Budapest Treaty. In general, the cells are grown in Dulbecco's modified Eagles medium (DMEM) supplemented with about 5% fetal bovine serum at 37° C. in the presence of about 5% CO₂. A test analyte suspected of being an HDAC inhibitor is then added to the medium and the cells incubated for a time sufficient for expression of β-lactamase. In general, incubating the cells for a time greater than 5 hours and preferably for a time greater than 13 hours in the presence of the analyte which is an HDAC inhibitor is sufficient to produce detectable activity. After sufficient time has been allowed to elapse for expression of the β-lactamase, the substrate is added comprising a cephalosporin cleavage site labeled with a donor:acceptor pair of fluorophores capable of FRET in the intact substrate which are situated on opposite sides of the cleavage site and detection of fluorescence produced by the donor fluorophore when the substrate is cleaved. Detection of the fluorescence can be by any means for detecting fluorescence. For example, the method can be performed using a 96-well microplate format with detection of substrate cleavage using a microplate reader such as an ELISA reader which is able to detect the fluorescence of the donor fluorophore.

In other aspects of the invention, the above incubation is performed in spinner cultures or shaker flasks using a variant of the above recombinant HeLa cell line which has been adapted for suspension culture using methods well-known in the art for adapting HeLa cells to suspension cultures or HeLa cells adapted for suspension (spinner) culture, for example HeLa S3 cells, which have been transfected with a plasmid vector comprising a reporter gene operably linked to a regulatory sequence activatable by an HDAC inhibitor, preferably under conditions that select stable transformants. In a currently preferred aspect, the reporter gene encodes β-lactamase and the reporter gene is operably linked to a regulatory sequence comprising nucleotides −185 to +25 flanking the p21 transcription start site (minimal p21 promoter). Substrate cleavage can be detected by any means suitable for detecting fluorescence including detection using a flow cytometry or fluorescence activated cell sorter (FACS).

The method of the present invention is particularly useful for high throughput screening (HTS) of analytes to identify analytes which are HDAC inhibitors. Often chemical entities with useful properties are generated by identifying a chemical compound (called a “lead compound”) with some desirable property or activity, creating variants of the lead compound, and evaluating the property and activity of those variant compounds. The current trend is to shorten the time scale for all aspects of drug discovery. Because of the ability to test large numbers quickly and efficiently, high throughput screening (HTS) methods are replacing conventional lead compound identification methods.

In one aspect, high throughput screening methods involve providing a library containing a large number of potential HDAC inhibitors. Such “combinatorial chemical libraries” are then screened in one or more assays, to identify those library members particular chemical species or subclasses that display a desired characteristic activity. The compounds thus identified can serve as conventional “lead compounds” or can themselves be used as potential HDAC inhibitors.

Devices for the preparation of combinatorial libraries are commercially available (See, for example, 357 MPS, 390 MPS, Advanced Chem Tech, Louisville, Ky.; Symphony, Rainin, Woburn, Mass.; 433A Applied Biosystems, Foster City, Calif.; 9050 Plus, Millipore, Bedford, Mass.). A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (See, for example, ComGenex, Princeton, N.J.; Asinex, Moscow, Russia; Tripos, Inc., St. Louis, Mo.; ChemStar, Ltd, Moscow, Russia; 3D Pharmaceuticals, Exton, Pa.; Martek Biosciences, Columbia, Md.).

Any of the assays described herein are amenable to high throughput screening. As described above, the HDAC inhibitors are preferably screened by the methods disclosed herein. High throughput systems for such screening are well known to those of skill in the art. Thus, for example, U.S. Pat. No. 5,559,410 discloses high throughput screening methods for protein binding, while U.S. Pat. Nos. 5,576,220 and 5,541,061 disclose high throughput methods of screening for ligand/antibody binding.

In addition, high throughput screening systems are commercially available (See, for example, Zymark Corp., Hopkinton, Mass.; Air Technical Industries, Mentor, Ohio; Beckman Instruments, Inc. Fullerton, Calif.; Precision Systems, Inc., Natick, Mass.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols. Thus, for example, Zymark Corp. provides technical bulletins describing screening systems for detecting the modulation of gene transcription, ligand binding, and the like.

The present invention further provides a method for treating a cancer in a patient. The method comprises providing one or more cultures of cells which include therein a gene expression cassette comprising a reporter gene operably linked to a transcription regulatory sequence wherein the transcription regulatory sequence includes nucleotide sequences responsive to a histone deacetylase (HDAC) inhibitor selected from the group consisting of Apicidin, Trichostatin A, sodium butyrate, SAHA, and MS27-275 stably integrated into the genome of the cells; culturing each of the one or more cultures of cells in a medium which contains an analyte; identifying the analytes which stimulate expression of the reporter gene in the cells; and then administering one or more of the analytes identified in the preceding step to stimulate expression of the reporter gene to the patient to treat the cancer. In further aspects, the gene expression cassette comprises the β-lactamase gene operably linked to the p21 minimal promoter. The method can use a cell line such as HeLa cells or the cells deposited as ICLC PD02008. In particular embodiments, the cells can include cells obtained from the patient. The cells are then transfected with the DNA comprising the gene expression cassette disclosed herein to perform the method disclosed herein.

In a further embodiment, the present invention provides a method for inducing differentiation or apoptosis of a proliferative cell, which comprises providing one or more cultures of cells which include therein a gene expression cassette comprising a reporter gene operably linked to transcription regulatory sequence which includes nucleotide sequences responsive to a histone deacetylase (HDAC) inhibitor selected from the group consisting of Apicidin, Trichostatin A, sodium butyrate, SAHA, and MS27-275 stably integrated into the genome of the cells; culturing each of the one or more cultures of cells in a medium which contains an analyte; identifying the analytes which stimulate expression of the reporter gene in the cells; and administering one or more of the analytes identified in the preceding step to stimulate expression of the reporter gene to the proliferative cell to induce the differentiation or apoptosis of the proliferative cell. In further aspects, the gene expression cassette comprises the β-lactamase gene operably linked to the p21 minimal promoter. The method can use a cell line such as HeLa cells or the cells deposited as ICLC PD02008. In particular embodiments, the cells can include particular tumor cells, stem cells, or the like. The cells are then transfected with the DNA comprising the gene expression cassette disclosed herein to perform the method disclosed herein.

While the examples provided herein use cells stably transfected with a gene expression cassette comprising a reporter gene operably linked to the p21 minimal promoter or a transcription regulatory sequence of any one of the aforementioned genes responsive to an HDAC inhibitor, the gene expression cassette can comprise a reporter gene operably linked to an HDAC subtype-responsive transcription regulatory sequence which has been identified using antisense oligonucleotides (ASO) or small interfering RNAs (siRNAs) that selectively inhibit the expression of a given HDAC subtype. Thus, cells stably transfected with a gene expression cassette comprising a reporter gene operably linked to a transcription regulatory sequence regulated by a particular HDAC subtype can be used to identify HDAC inhibitors that activate transcription of genes deactivated by particular HDAC subtypes.

In general, cultured cells are transfected with ASO or an siRNA that inhibit the expression of a given histone deacetylase. Standard transfection such as CaPO4 precipitation, electroporation, DEAE-dextran, oligofectamin, lipofectamin, lipofectin, or the like can be used. Upon transfection, cells are cultured for a time suitable to decrease the expression levels of the targeted HDAC. This time will depend on the stability (half life) of the given HDAC subtype and on the cell line and knock-down technique used. Protein expression levels can be monitored by Western blot or ELISA and can be used to assess the knock-down efficacy. Multiple, repeated transfections may be required to optimize the downregulation. Also, transfections using mixtures of different ASOs or siRNAs targeting different regions of the same mRNA may be used. A preferred condition would achieve a 2 to 10-fold decrease of the target protein content of cells treated with ASO or siRNAs, but higher degrees of knock-down may be obtained and would be more preferable. Control ASO or siRNAs can be used which contain one or more mismatches with respect to their target sequence. Mismatch oligos can be transfected in the same way but will not affect the target protein expression levels.

Cells in which the target HDAC expression levels were decreased by the action of an ASO or an siRNA as well as cells which were grown for the same amount of time upon transfection with a mismatch control ASO or siRNA are collected and total RNA extracted. RNA extraction can be performed using commercially available kits (RNeasy, Qiagen; Trizol, Invitrogen Life Techonolgies), following the manufacturer's instructions. Alternatively, published methods (Chomczynskiand Sacchi, Anal. Biochem. 162: 156 (1987); Sambrook et al. ) can be used.

In order to identify genes that are specifically regulated by a given HDAC, the amount of mRNA generated from any particular gene is quantified. To this purpose, RNA extracted from cells in which the expression of a particular HDAC subtype was decreased by transfection with the appropriate ASOs or siRNAs and RNA extracted from cells treated with mismatch control ASOs or siRNAs is subjected to further analysis. If several genes are to be analysed at the same time, their expression levels can be quantified by techniques such as Northern blot analysis, RT-PCR, or Taqman RT-PCR. If a large number of genes are to be analyzed at the same time or if all genes that are affected in their expression by a particular HDAC are to be identified at the same time, microarray analysis can be used.

To this purpose, the RNA extracted from untreated, mock-transfected cells, cells transfected with specific siRNAs or ASOs that cause a decrease in the expression levels of the targeted HDAC, or cells that were transfected with mismatch control siRNAs or ASOs are labeled and hybridized to an array of immobilized complementary oligonucleotides. Preferably, the array contains a number of oligonucleotides which includes all human genes. However, arrays allowing the identification of only hundreds or thousands of genes are also useful. The labeled RNA (target) will specifically anneal with its immobilized, complementary oligonucleotide of known sequence (probe). Each oligonucleotide probe is located in a specific area of the array (probe cell). The method can be used to quantify the amount of a particular mRNA since the amount of label captured by the oligonucleotide probe for the particular mRNA contained in the array will be proportional to the amount of the particular mRNA in the RNA extract. The amount can be quantified using an appropriate instrumentation which can detect the label. For example, if the label is fluorescent, fluorescence emission intensity can be quantified over the whole array. Labels with different fluorescence properties can be used to label RNA populations from control and treated cells or derivatives thereof. Methods for labeling RNA are known to those skilled in the art. For example, fluorescent labeling of an RNA can be achieved by introducing a biotin label into the RNA population using the methodology described below and staining the labeled RNA population using a streptavidin phycoerythrin conjugate. An oligodT primer is added to 5 to 40 μg of isolated RNA to prime the reverse-transcription of the polyA+RNA molecules. This produces a double-stranded RNA-DNA hybrid molecule. The RNA component of this molecule can be digested using RNAseH and a second DNA strand can be synthesized using a DNA-dependent DNA polymerase such as T4 DNA polymerase. The resulting dsDNA can be used to synthesize a conveniently labeled cRNA probe. Commercial kits such as the ENZO bioarray High yield RNA transcript labeling kit (Affymetrix cod 900182) can be used to produce the labeled cRNA probe. The label is introduced into the cRNA by using modified nucleotides containing the label in the reaction mix. The resulting labeled cRNA molecules are then fragmented by controlled DNase digestion to yield fragments between about 35 to 200 nucleotides. These fragments can be directly hybridized to an array such as the GeneChip probe array (Affymetrix) and reacted with streptavidin phycoerythrin followed by removal of unreacted fluorophore. To obtain expression data, the array is subsequently scanned by exciting the fluorophore at the appropriate wavelength. If phycoerythrin is used, the optimal excitation wavelength is 488 nm, resulting in an emission of fluorescence at 570 nm. The fluorescence intensity can be automatically recorded with the appropriate instrumentation and software such as Affymetrix Microarray Suite or Rosetta Resolver.

Analysis of gene expression patterns obtained using this or similar technologies can be used to define the set of genes that are regulated by a particular HDAC subtype. The transcription regulatory sequence for any one of the genes identified to be regulated by a particular HDAC subtype can be operably linked to a reporter gene as described herein to make a gene expression cassette for producing cells with the expression cassette stably integrated into the genome thereof. For example, cells expressing HDAC1 are transfected with ASOs or siRNAs targeting the mRNA encoding the HDAC1. This causes a decrease in the amount of HDAC1 expressed in the transfected cell and an increase in the expression of genes whose expression is normally repressed by HDAC1. Gene expression analysis of the RNA extracted from the transfected cells using microarray technology identifies the genes whose expression are controlled by HDAC1. The HDAC1 responsive transcription regulatory sequence is operably linked to a reporter gene to produce a gene expression cassette which is then transfected into a cell to produce a recombinant cell comprising the gene expression cassette. The recombinant cell is used as described herein to identify analytes which are inhibitors of HDAC1 activity or ability to inhibit expression of the gene.

The following examples are intended to promote a further understanding of the present invention.

### EXAMPLE 1

A plasmid vector was constructed comprising a gene expression cassette consisting of the human p21 minimal promoter operably linked to a reporter gene encoding β-lactamase. The p21 minimal promoter was cloned into the pcDNA3-BlaM-Neo reporter plasmid vector (Aurora Biosciences Corp., San Diego, Calif.) to produce the plasmid vector p21m-BLA. The p21 minimal promoter includes nucleotides −183 to +25 flanking the p21^{WAF1/CiP1} gene transcription start site as shown in SEQ ID NO:1. The pcDNA3-BlaM-Neo plasmid vector contains the CMV promoter operably linked to the BlaM gene which is replaced with the minimal p21 promoter. BlaM is a Bla gene which had been modified for expression in mammalian cells.

To isolate the p21 minimal promoter, the PAC genomic clone RP-1 193M11 (Gen Bank accession No. AL121715, available from the Wellcome Trust Sanger Institute, Wellcome Trust Genome Campus, Hixton, Cambridge, UK and Children's Hospital Oakland, BACPAC Resources, Bruce Lyon Memorial Research Building, Oakland, Calif. (Peter J. de Jong)) containing approximately 94 kb of DNA upstream of the p21^{WAF1/CiP1} transcriptional start site and 47 kb downstream of the p21^{WAF1/CiP1} transcriptional start site was obtained. A 19 kb restriction enzyme DNA fragment spanning the entire 5′ regulatory region and 1.1 Kb of the transcribed region of the p21^{WAF1/CiP1} was sub-cloned in a suitable plasmid. The p21 minimal promoter was amplified by PCR using a forward primer having a BglII site at the 5′end corresponding to position −183 and a reverse primer having a KpnI site at the 3′ end corresponding to position +25. The PCR primers and conditions for p21 minimal promoter amplification were as follows. The forward primer was 5′-TAA CGG AAG ATC TTG CTG GAA CTC GGC CAG GCT CAG C-3′ (SEQ ID NO:2) and the reverse primer was 5′-ACT CGG TGG TAC CAA GCT TGG CTC CAC AAG GAA CTG ACT TCG GC-3′ (SEQ ID NO:3). The BglII site in the forward primer and the KpnI site in the reverse primer are underlined. The PCR amplifications used the TaKaRaLa Taq polymerase (TaKaRa Bio. Europe S.A., Gennevilliers, France: catalog number: RR002A) and followed the conditions suggested by the manufacturer plus adding 5% DMSO. Reactions were cycled as follows: 96° C. for two minutes, followed by 25 cycles at 96° C. for 30 seconds, 60° C. for 15 seconds, 72° C. for 60 seconds for each cycle.

The amplified minimal promoter was digested with BglII and KpnI and then cloned into pcDNA3-BlaM-Neo vector (Aurora Biosciences Corporation, La Jolla, Calif.) which had been digested with BglII and KpnI to remove the CMV promoter operably linked to the BlaM gene to produce the p21m-BLA reporter construct (FIG. 2A). The nucleotide sequence of plasmid p21m-BLA (SEQ ID NO:4) is shown in FIG. 2B through 2D. The sequence of the p21 minimal promoter (SEQ ID NO:1) is shown in FIG. 1. The p21 minimal promoter fragment replaces the entire CMV promoter of the pcDNA3-BlaM-Neo plasmid. The plasmid vector with the p21 minimal promoter ligated therein (p21m-BLA) was then transformed into E. coli.

A plasmid containing a gene expression cassette comprising the gene encoding secreted alkaline phosphatase (SEAP) operably linked to the p21 minimal promoter (p21m-SEAP) was constructed as follows. A nucleotide fragment (about 1816 bp) containing the entire open reading frame (ORF) encoding the SEAP was removed from plasmid pSEAP2 Basic (commercially available from Clontech, San Diego, Calif., catalog # 6049-1) by digesting the plasmid with SalI, filling in the cleaved SalI ends with Klenow polymerase to make the SalI ends blunt, digesting the plasmid with HindIII, and separating the 1816 bp nucleotide fragment encoding the SEAP ORF with a blunt SalI end and a HindIII overhang end was separated from the plasmid. The entire ORF encoding the β-lactamase (about 810 bp) was removed from p21m-BLA by digesting the plasmid with XbaI, filling in the cleaved XbaI ends with Klenow polymerase to make the XbaI ends blunt, digesting the plasmid with HindIII, and separated the XbaI (blunt)/HindIII digested plasmid from the 810 bp nucleotide fragment encoding the β-lactamase. The SalI (made blunt ended)/HindIII nucleotide fragment encoding the SEAP ORF was then ligated between the XbaI (blunt)/HindIII sites of the p21m-BLA plasmid to produce p21m-SEAP.

All DNA manipulations and cloning steps were carried out employing standard molecular biology techniques known to those skilled in the art (For example, see Sambrook et al., Molecular Cloning: A Laboratory Manual 2nd Edition; Cold Spring Harbor Laboratory Press, Cold Spring Harbor, N.Y., (1989) or Sambrook and Russell, Molecular Cloning: A Laboratory Manual, 3rd Edition. Cold Spring Harbor Laboratory Press, Plainview, N.Y. (2001)).

### EXAMPLE 2

The p21m-BLA reporter plasmid was tested in transient transfection experiments of HeLa cells to evaluate the responsiveness of the minimal p21 promoter and the Bla reporter system to known HDAC inhibitors. In the first two transfection experiments, HeLa cells were transfected with 1 or 2 μg of Bla reporter plasmid containing either p21 minimal promoter (p21m-BLA) or CMV promoter (CMV-BLA) and 0.1 μg of pCMV-Luc or pRSV-Luc control plasmids. Different DNA-FUGENE transfection reagent ratios were tested (FUGENE is available from Roche Applied Science, Indianapolis, Ind.). Five hours post-transfection, fresh medium was added containing either DMSO or 400 nM TSA and the cells were analyzed after 12 hours induction both for Bla and Luc activities. In both experiments, approximately 50% of the cells were dead after TSA treatment. Data are summarized below in Tables 1 and 2.

TABLE 1

| | | | | Fold |
|---|---|---|---|---|
| Transfected Plasmids and | | % Blue | RLU/ | induction |
| FUGENE (F) | Compound | cells | mg | (RLU) |
| 1 μg p21m-BLA + 3 μL F | DMSO | 2-3 | 3245.4 | — |
| 2 μg p21m-BLA + 6 μL F | | 4 | 7836.4 | — |
| 1 μg p21m-BLA + 5 μL F | | 3 | 1987.3 | — |
| 2 μg p21m-BLA + 10 μL F | | 3-4 | 2829 | — |
| 1 μg BLA + 3 μL F | | 10-15 | 495 | — |
| 2 μg BLA + 6 μL F | | 35 | 2453 | — |
| 1 μg p21m-BLA + 3 μL F | 400 nM | 1 | 52035 | 16 |
| 2 μg p21m-BLA + 6 μL F | TSA | 1 | 50773.9 | 6.5 |
| 1 μg p21m-BLA + 5 μL F | | <1 | 29022.4 | 14.6 |
| 2 μg p21m-BLA + 10 μL F | | <1 | 22499.2 | 7.95 |
| 1 μg CMV-BLA + 3 μL F | | 5 | 8986 | 18 |
| 2 μg CMV-BLA + 6 μL F | | 15-20 | 14934 | 6 |

TABLE 2

| Transfected Plasmids and | | | | Fold induction |
|---|---|---|---|---|
| FUGENE (F) | Compound | % Blue cells | RLU/mg | (RLU) |
| 1 μg p21m-BLA + 3 μL F | DMSO | 5 | — | — |
| 2 μg p21m-BLA + 6 μL F | | 5-10 | — | — |
| 1 μg p21m-BLA + 3 μL F + RSV-luc | | 5-10 | 71.7 | — |
| 2 μg p21m-BLA + 6 μL F + RSV-luc | | 10 | 404.15 | — |
| 1 μg p21m-BLA + 3 μL F + CMV-luc | | 8 | 2864 | — |
| 2 μg p21m-BLA + 6 μL F + CMV-luc | | 10-15 | 8322.65 | — |
| 1 μg p21m-BLA + 3 μL F | 400 nM | 5 | — | — |
| 2 μg p21m-BLA + 6 μL F | TSA | 5 | — | — |
| 1 μg p21m-BLA + 3 μL F + RSV-luc | | 5 | 24437.8 | 340.8 |
| 2 μg p21m-BLA + 6 μL F + RSV-luc | | 4 | 29989.4 | 74 |
| 1 μg p21m-BLA + 3 μL F + CMV-luc | | 4 | 47709.8 | 16.7 |
| 2 μg p21BLA + 6 μL F + CMV-luc | | 4 | 105942 | 12.7 |
| 1 μg CMV-BLA + 3 μL F | DMSO | 15 | — | — |
| 2 μg CMV-BLA + 6 μL F | | 25-30 | — | — |
| 1 μg CMV-BLA + 3 μL F + RSV-luc | | 15 | 143.6 | — |
| 2 μg CMV-BLA + 6 μL F + RSV-luc | | 30 | 275 | — |
| 1 μg CMV-BLA + 3 μL F + CMV-luc | | 15 | 1777.7 | — |
| 2 μg CMV-BLA + 6 μL F + CMV-luc | | 30 | 5891.8 | — |
| 1 μg CMV-BLA + 3 μL F | 400 nM | 5 | — | — |
| 2 μg CMV-BLA + 6 μL F | TSA | 5 | — | — |
| 1 μg CMV-BLA + 3 μL F + RSV-luc | | 7 | 7964.46 | 55.4 |
| 2 μg CMV-BLA + 6 μL F + RSV-luc | | 7 | 4972 | 18 |
| 1 μg CMV-BLA + 3 μL F + CMV-luc | | 5 | 14928.8 | 8.4 |
| 2 μg CMV-BLA + 6 μL F + CMV-luc | | 7 | 13085.6 | 2.2 |

In both transient transfection experiments, normalized Luc values were comparable in cells co-transfected with either p21m-BLA or pCMV-BLA and treated with DMSO, thus suggesting that the efficiency of transfection was similar. However, the percentage of Bla-positive cells was lower with p21-Bla reporter than with pCMV-BLA reporter. The fraction of Bla-positive cells treated with TSA was equal or lower than that measured in DMSO with both Bla reporters maybe because of a selective loss of positive cells due to cell death. Nevertheless, in the same cells, luciferase expression driven by both CMV and RSV promoters was induced by TSA to various extents. The lack of evidence for TSA-dependent p21m-BLA (and pCMV-BLA) induction might have been due to expression of saturating amounts of the enzyme in the presence of DMSO or to a lack of activation in the residual living cells. This might have been due to a selective loss of Bla-expressing cells after TSA treatment, an insufficient accumulation of β-lactamase during the time of induction, an incubation time between transfection and addition of the HDAC inhibitor inadequate to allow binding of nucleosomes to the transfected plasmid and consequent HDAC-mediated repression of transcription, or malfunctioning of the p21 minimal promoter.

In a further transient transfection experiment, different times (6 hours, 12 hours, 24 hours) of treatment were compared with either TSA (300 nM) or Apicidin (1 μM) allowing a longer time span (24 hr) between transfection and addition of HDAC inhibitors. The results are presented in Table 3. Cell-death was not visible after 6 hours whereas a higher fraction of cells died at longer incubation times in the presence of TSA than in the presence of Apicidin. The percentage of β-lactamase-positive cells increased with time independent of the presence of HDAC inhibitors and was constantly higher with p21m-BLA than with pCMV-BLA. Cell inspection also at shorter time-points after the addition of BLA substrate failed to reveal substantial differences of blue color intensity between treated and untreated cells. The relatively high percentage of blue cells measured in this experiment in cells transfected with p21m-BLA corresponded to a significantly high basal expression of pCMV-Luc in the same cells.

TABLE 3

| Transfected | | | | | |
|---|---|---|---|---|---|
| Plasmids | | | | | Fold |
| (2 μg + 0.1 μg | | Time of | % Blue | | Ind. |
| pCMV-Luc) | Compound | treatment | cells | RLU/mg | RLU |
| p21m-BLA | DMSO | 6 hr | 5 | 21878.6 | — |
| p21m-BLA | 300 nM TSA | 6 hr | 2-3 | 85379 | 3.9 |
| p21m-BLA | 1 μM Apic. | 6 hr | 6 | 79723 | 3.6 |
| pCMV-BLA | DMSO | 6 hr | 3 | 5103 | — |
| pCMV-BLA | 300 nM TSA | 6 hr | 3_4 | 12579.8 | 2.5 |
| pCMV-BLA | 1 μM Apic. | 6 hr | 4 | 10937 | 2 |
| p21m-BLA | DMSO | 12 hr | 15-20 | 75004.4 | — |
| p21m-BLA | 300 nM TSA | 12 hr | 10 | 248656.9 | 3.3 |
| p21m-BLA | 1 μM Apic. | 12 hr | 10 | 183519 | 2.45 |
| pCMV-BLA | DMSO | 12 hr | 5-6 | 6959 | — |
| pCMV-BLA | 300 nM TSA | 12 hr | 4-5 | 33592.3 | 2.7 |
| pCMV-BLA | 1 μM Apic. | 12 hr | 3 | 39603.5 | 3.6 |
| p21m-BLA | DMSO | 24 hr | 25 | 45177.7 | — |
| p21m-BLA | 300 nM TSA | 24 hr | 10 | 110180.6 | 2.4 |
| p21m-BLA | 1 μM Apic. | 24 hr | 25 | 131733 | 2.9 |
| pCMV-BLA | DMSO | 24 hr | 10-15 | 3591.7 | — |
| pCMV-BLA | 300 nM TSA | 24 hr | 15 | 22233.7 | 6 |
| pCMV-BLA | 1 μM Apic. | 24 hr | 10 | 18932.5 | 5.3 |

To determine whether the p21 minimal promoter was responsive to HDAC inhibitors, a reporter plasmid containing the secreted alkaline phosphatase gene (SEAP) operably linked to the p21 minimal promoter (−182 through +25), pcDNA3-21m-SEAP, was tested in transient transfection experiments of HeLa cells. A promoter titration experiment was carried out in the presence or absence of Apicidin (1 μM) and the result shows a curve of induction (Table 4). All data summarized in Tables 4 through 7 were obtained by normalizing the total amount of transfected DNA to 2.1 μg using a promoterless plasmid.

TABLE 4

| | P21m- | | | | SEAP | |
|---|---|---|---|---|---|---|
| | SEAP | SEAP | Fold | Tot. prot. | norm | Fold |
| Inducer | (μg DNA) | (10 min) | induct. | (mg/mL) | (mg/mL) | Induct. |
| 1.1 DMSO | 0 | 3247 | — | 2.8 | 1159.643 | — |
| 1.2 DMSO | 0.1 | 8587 | — | 2.4 | 3577.917 | — |
| 1.3 DMSO | 0.2 | 3247 | — | 2.9 | 1119.655 | — |
| 1.4 DMSO | 0.5 | 8485 | — | 3 | 2828.333 | — |
| 1.5 DMSO | 1 | 9734 | — | 2.8 | 3476.429 | — |
| 1.6 DMSO | 2 | 80823 | — | 2.8 | 28865.36 | — |
| 2.1 Apicidin (1 μM) | 0 | 11366 | 3.5 | 2 | 5683 | 4.90065 |
| 2.2 Apicidin (1 μM) | 0.1 | 41278 | 4.8 | 1.5 | 27518.67 | 7.69125 |
| 2.3 Apicidin (1 μM) | 0.2 | 33449 | 10.3 | 2 | 16724.5 | 14.9372 |
| 2.4 Apicidin (1 μM) | 0.5 | 81729 | 9.6 | 1.5 | 54486 | 19.2643 |
| 2.5 Apicidin (1 μM) | 1 | 85405 | 8.8 | 1.4 | 61003.57 | 17.5478 |
| 2.6 Apicidin (1 μM) | 2 | 795345 | 9.8 | 1.8 | 441858.3 | 15.3076 |

Repetition of the experiment with Apicidin (1 μM) or TSA (300 nM) confirmed this result (Table 5).

TABLE 5

| | P21m- | | | | SEAP | | | |
|---|---|---|---|---|---|---|---|---|
| | SEAP | | | SEAP | norm.-bckg/ | | | RLU |
| | (μg | SEAP | SEAP | norm-bckg/ | RLU norm | Tot prot | | norm |
| Inducer | DNA) | (10 min) | (fold ind) | RLU norm | (fold ind) | (mg/ml) | RLU/μg | mean |
| 1.1 DMSO | 0 | 1206 | — | — | — | 1 | 34,213.60 | 1.11 |
| 1.2 DMSO | 0.1 | 1706 | — | 443.57 | — | 1 | 34,816.20 | 1.13 |
| 1.3 DMSO | 0.2 | 2740 | — | 612.08 | — | 1.5 | 31,320.53 | 1.01 |
| 1.4 DMSO | 0.5 | 2560 | — | 1,352.69 | — | 1.2 | 21,174.50 | 0.69 |
| 1.5 DMSO | 1 | 7162 | — | 4,034.38 | — | 1.4 | 29,932.57 | 0.97 |
| 1.6 DMSO | 2 | 24218 | — | 12,704.67 | — | 1.6 | 33,866.56 | 1.10 |
| 2.1 Apicidin | 0 | 2133 | 1.8 | — | — | 0.55 | 102,236.00 | 1.08 |
| (1 μM) | | | | | | | | |
| 2.2 Apicidin | 0.1 | 5661 | 3.3 | 4,740.65 | 10.7 | 0.6 | 111,192.67 | 1.17 |
| (1 μM) | | | | | | | | |
| 2.3 Apicidin | 0.2 | 10279 | 3.75 | 10,064.04 | 16.4 | 0.67 | 108,052.99 | 1.14 |
| (1 μM) | | | | | | | | |
| 2.4 Apicidin | 0.5 | 16014 | 6.25 | 24,280.99 | 17.9 | 0.9 | 54,363.72 | 0.57 |
| (1 μM) | | | | | | | | |
| 2.5 Apicidin | 1 | 48928 | 6.8 | 68,122.56 | 16.9 | 0.6 | 108,153.75 | 1.14 |
| (1 μM) | | | | | | | | |
| 2.6 Apicidin | 2 | 108562 | 4.5 | 219,589.86 | 17.3 | 0.54 | 85,172.59 | 0.90 |
| (1 μM) | | | | | | | | |
| 3.1 TSA | 0 | 2313 | 1.9 | — | — | 0.5 | 94,973.90 | 0.97 |
| (300 nM) | | | | | | | | |
| 3.2 TSA | 0.1 | 4405 | 2.6 | 6,558.72 | 14.8 | 0.4 | 95,526.00 | 0.97 |
| (300 nM) | | | | | | | | |
| 3.3 TSA | 0.2 | 9328 | 3.4 | 11,855.92 | 19.4 | 0.5 | 116,091.50 | 1.18 |
| (300 nM) | | | | | | | | |
| 3.4 TSA | 0.5 | 15743 | 6.1 | 25,471.82 | 18.8 | 0.6 | 83,237.58 | 0.85 |
| (300 nM) | | | | | | | | |
| 3.5 TSA | 1 | 50626 | 7.1 | 108,713.07 | 26.95 | 0.5 | 87,194.70 | 0.89 |
| (300 nM) | | | | | | | | |
| 3.6 TSA | 2 | 109998 | 4.5 | 201,678.57 | 15.9 | 0.47 | 111,592.45 | 1.14 |
| (300 nM) | | | | | | | | |

The results show a significant induction of SEAP for both HDAC inhibitors (up to 20 fold for Apicidin and up to 26 fold for TSA). The results further show that the p21 minimal promoter is responsive to HDAC inhibitors.

In light of the above, the lack of apparent Bla activation in the above transfection experiments might have been due to the high sensitivity of the method. Therefore, transient transfections in which an accurate titration of the reporter plasmid DNA were performed. The titration of the reporter plasmid included using amounts lower than was used above. As shown in Table 6, transient transfections of HeLa cells and incubation in the presence or absence of Apicidin (1 μM) resulted in a 3- to 5-fold induction of β-lactamase activity only in the treated samples transfected with the lowest DNA amounts (0.02 and 0.05 ng). The transient transfections were repeated and the result confirmed (Table 7).

TABLE 6

| | | | Bla | fold-ind. |
|---|---|---|---|---|
| | pCMV-BLA | p21m-BLA | staining | at 60 min. |
| Inducer | (μg DNA) | (μg DNA) | at 60 min. | (Bla) |
| 1.1 DMSO | 0 | — | 0 | — |
| 1.2 DMSO | 0.02 | — | 5% | — |
| 1.3 DMSO | 0.05 | — | 5-10% | — |
| 1.4 DMSO | 0.1 | — | 10-15% | — |
| 1.5 DMSO | 0.2 | — | 20% | — |
| 1.6 DMSO | 0.5 | — | 40% | — |
| 2.1 Apicidin (1 μM) | 0 | — | 0 | — |
| 2.2 Apicidin (1 μM) | 0.02 | — | 5% | 1 |
| 2.3 Apicidin (1 μM) | 0.05 | — | 5-10% | 1 |
| 2.4 Apicidin (1 μM) | 0.1 | — | 10-15% | 1 |
| 2.5 Apicidin (1 μM) | 0.2 | — | 20% | 1 |
| 2.6 Apicidin (1 μM) | 0.5 | — | 40% | 1 |
| 3.1 DMSO | — | 0 | 0 | — |
| 3.2 DMSO | — | 0.02 | 0.5-1% | — |
| 3.3 DMSO | — | 0.05 | 2-3% | — |
| 3.4 DMSO | — | 0.1 | 15% | — |
| 3.5 DMSO | — | 0.2 | 20% | — |
| 3.6 DMSO | — | 0.5 | 40% | — |
| 4.1 Apicidin (1 μM) | — | 0 | 0 | — |
| 4.2 Apicidin (1 μM) | — | 0.02 | 2-3% | 3-4 |
| 4.3 Apicidin (1 μM) | — | 0.05 | 10-15% | 5 |
| 4.4 Apicidin (1 μM) | — | 0.1 | 25-30% | 2 |
| 4.5 Apicidin (1 μM) | — | 0.2 | 35-40% | 2 |
| 4.6 Apicidin (1 μM) | — | 0.5 | 50% | 1.25 |

TABLE 7

| | | | Bla | fold-ind. |
|---|---|---|---|---|
| | pCMV-BLA | p21m-BLA | staining | at 15 min. |
| Inducer | (μg DNA) | (μg DNA) | at 15 min | (Bla) |
| 1.1 DMSO | 0 | — | 0 | — |
| 1.2 DMSO | 0.02 | — | 3-5% | — |
| 1.3 DMSO | 0.05 | — | 10% | — |
| 1.4 DMSO | 0.1 | — | 15% | — |
| 1.5 DMSO | 0.2 | — | 25-30% | — |
| 1.6 DMSO | 0.5 | — | 40% | — |
| 2.1 DMSO | — | 0 | 0 | — |
| 2.2 DMSO | — | 0.02 | 3% | — |
| 2.3 DMSO | — | 0.05 | 5% | — |
| 2.4 DMSO | — | 0.1 | 15% | — |
| 2.5 DMSO | — | 0.2 | 30% | — |
| 2.6 DMSO | — | 0.5 | 40% | — |
| 3.1 Apicidin (1 μM) | — | 0 | 0 | — |
| 3.2 Apicidin (1 μM) | — | 0.02 | 6-10% | 2-3 |
| 3.3 Apicidin (1 μM) | — | 0.05 | 20-25% | 4-5 |
| 3.4 Apicidin (1 μM) | — | 0.1 | 25-30% | 2 |
| 3.5 Apicidin (1 μM) | — | 0.2 | 40% | 1.3 |
| 3.6 Apicidin (1 μM) | — | 0.5 | 40% | 1 |

The results suggest that copy number of the reporter plasmid in stably transfected cells is an important consideration, particularly when the reporter gene is of a type which enables the stably transfected cells to be sensitive to HDAC inhibitors. For example, as suggested herein, stably transfected cells containing a low copy number of the p21m-BLA will provide cells which are sensitive to the presence of HDAC inhibitors and which are useful in assays for identifying analytes which are HDAC inhibitors whereas stably transfected cells containing a large copy number of p21m-BLA might have reduced sensitivity to HDAC inhibitors or no sensitivity at all.

### EXAMPLE 3

To increase the probability of selecting stably transfected cell clones containing the p21m-Bla in a single copy number, conditions were optimized to introduce p21m-BLA by electroporation in HeLa cells which do not contain functional p53. Plasmid pcDNA3-BlaM was used as a test plasmid to identify conditions in which cell viability was high. The efficiency of transfection evaluated upon BLA-staining was approximately 25% and different shades of blue were visible possibly indicating the presence of different copy numbers of the pcDNA-BlaM in different cells.

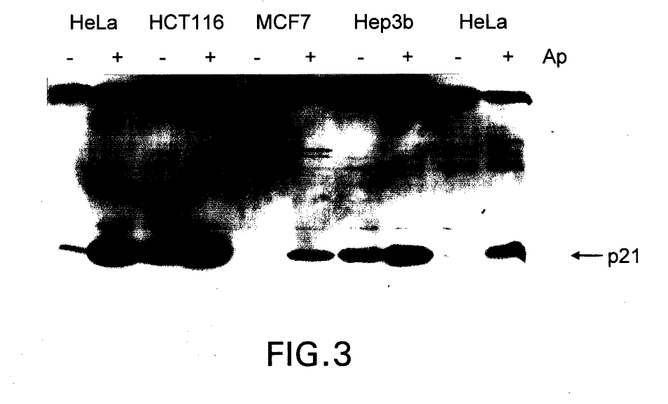

HeLa cells were chosen for transfection because they showed the best ratio between induced endogenous p21 protein expression and background p21 protein expression when compared to the human tumor cell lines HCT116, MCF7, and Hep3b (FIG. 3). In addition, HeLa cells lack functional p53 which reduces p21^{WAF1/CIP1} promoter basal activity as compared with cell lines which have a functional p53. All cells were grown in complete DMEM: Dulbecco's Modified Eagle Medium (DMEM, GIBCO Invitrogen, Carlsbad, Calif.) containing 0.11 g/L Pyridoxine and complemented with L-Glutamine (200 mM, GIBCO) at a final concentration of 2 mM, Penicillin-Streptomycin (10 mg/ml, GIBCO) at a final concentration of 0.1 mg/ml, and 10% (v/v) Fetal Calf Serum (FCS, GIBCO). Geneticin (G418) was added to the medium at a final concentration of 0.45 mg/ml for selection and maintenance of stable transformants.

The transfections were as follows. Adherent HeLa cells in log phase, about 80% confluent (total cells about 26×10⁶) on tissue culture plates, were trypsinized, diluted in complete cold DMEM, and removed from the plate. The cells were pelleted by low speed centrifugation (about 250×g for 5 minutes at 4° C.) and the cell pellet resuspended in cold complete medium (DMEM) to a final concentration of about 10×10⁶ cells/mL. About 2×10⁶ cells were mixed with 10 μL of p21m-BLA DNA at 1 μg/μL in 0.2 cm cuvettes, chilled on ice, and then electroporated at 950 μF and 80 Volts in a GENE PULSER II electroporation apparatus (BioRad Laboratories, Hercules, Calif.). Three electroporations, each containing about 2×10⁶ cells, were carried out, obtaining slightly different time constants of 32.2, 31.9, and 33.9 milliseconds, respectively. Subsequently cells were diluted to 1.5×10⁵ per mL in cold complete DMEM and each of the three electroporated samples plated at 1.5×10⁶ per culture dish (10 cm diameter). After about 4 hours, about 35 to 40% of the electroporated cells had attached to the tissue plate surface. One mL of cells for each electroporation was also plated in duplicate in six-well tissue culture plates in order to check transfection efficiency. Random integration frequencies have been reported in the literature to be about 0.1%.

After about 24 hours, the medium was changed to remove cell debris. After 24 hours, the electroporated cells were about 80% confluent and the electroporated cells in the six-well plates were about 40% confluent. About 48 hours post-electroporation, the electroporated cells on the 10 cm plates were trypsinized, centrifuged at low speed to pellet the cells, and the pellets resuspended in complete DMEM and pooled. The total number of cells was 19.2×10⁶ which suggested a regular doubling time for the 48 hour period and 4.5×10⁶ cells. The cells were split onto thirty 10 cm tissue culture plates at a dilution of about 1:15, thirty 10 cm plates at a dilution of 1:50, and four 10 cm plates at a dilution of 1:10. Cells were first diluted in 45 mL of cold non-selective complete medium and 1 mL aliquots were plated onto each of the 10 cm plates for the 1:15 dilutions. Then 35 mL of medium was added to the remaining cells to make a 1:50 dilution and 1 mL aliquots were plated onto each of thirty 10 cm plates. Finally, the remaining 20 mL was split onto four 5 mL aliquots and each plated onto a 10 cm plate. Then for all plates, Geneticin (G418; 80 mg/mL) was added to give a final concentration of about 450 μg/mL. Medium for each plate was changed every three days. After several weeks in selective medium, about 130 independent clones were collected and expanded. One hundred-eleven stable amplified transformants were each trypsinized from their plates, centrifuged at low speed to pellet the cells, and the cell pellet resuspended in complete DMEM.

All clones were screened for the presence of stably and functional integrated copies of the p21m-BLA reporter gene transcription cassette by performing a fluorometric β-lactamase activity assay after treatment with the HDAC inhibitor Apicidin at a concentration of 1 μM (saturating dose representing 100% of Bla gene activation, i.e., 100% of HDAC inhibition). A 100 μL aliquot of cells for each stable transformant in complete DMEM was plated into a well of a 96 well plate at about 36,000 cells per well for treatment with 1 μM Apicidin (saturating dose) and a 100 μL aliquot of cells was plated into a well of a 96 well plate at 24,000 cells per well for treatment with DMSO (vehicle control). Each plate also had four DMEM controls and four HeLa cell controls prepared in the same manner. After 4 hours, 25 μL of complete DMEM containing Apicidin at 5× final concentration or DMSO (5%) was added to each of the wells. The cells were then grown at 37° C., 5% CO₂ for about 12 to 16 hours. Afterwards, the medium was removed and 100 μL of DMEM without serum and 25 μL of FRET labeled CCF2-AM Bla substrate diluted in the appropriate buffer according to the manufacturer recommendations were each added to the cells to give a final concentration of 1 μM CCF2-AM. Fluorescence was read at λₑₓ 405/20 λₑₘ 460/40 on a FLUOSTAR (BMG Labtechnologies, Offenburg Germany).

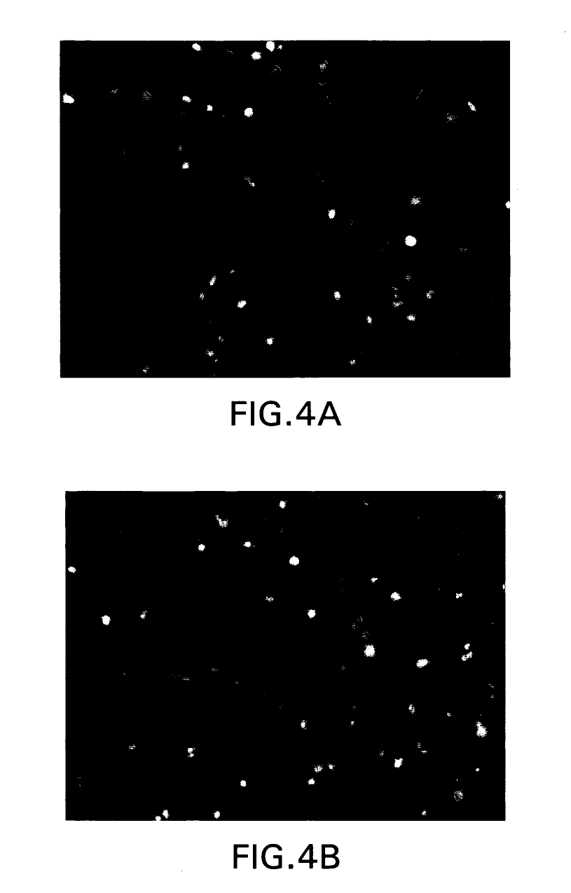

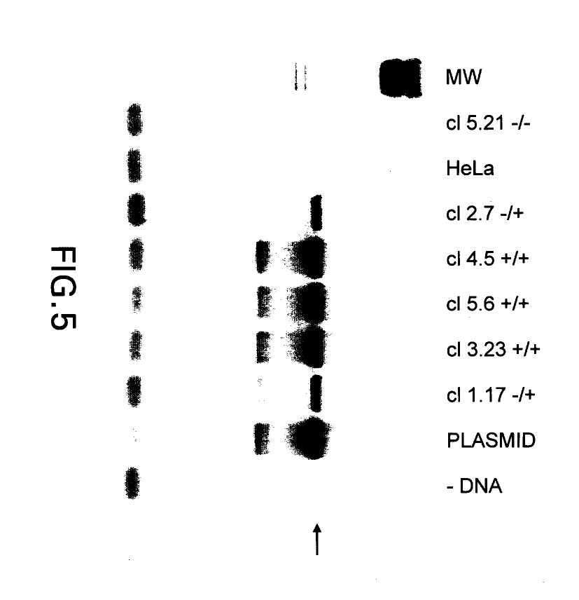

About 40% of the transformants showed constitutive expression of β-lactamase activity. Only two clones, 1.17 and 2.7 had both good inducibility with Apicidin and low background. The remaining clones were either G418-resistant and did not respond to Apicidin or had low levels of inducibility. Slope analysis of the best performing clone, 1.17, in the absence or presence of saturating amounts of Apicidin showed roughly a 200-fold induction over DMSO (FIG. 4).

Clone 1.17 was subjected to a first fluorescence activated cell sorter (FACS) sorting cycle after treatment with 600 nM TSA for 13 hr and β-lactamase (Bla) staining. Blue-positive cells were collected and after 7 days in culture challenged with 1 μM Apicidin or DMSO for 13 hours. Complete reversion of TSA-induced Bla induction was observed in samples treated with DMSO but no apparent enrichment in the number of blue-positive cells was obtained in Apicidin-treated samples after this procedure. A second FACS sorting cycle was repeated on these cells cultured in the absence of the inhibitor and treated for Bla staining.

Analysis of the β-lactamase-negative cells collected after the second FACS sorting cycle of clone 1.17 did not show any apparent decrease of β-lactamase background activity. Therefore, it was concluded that the heterogeneity observed in the clonal cell population both in the absence and presence of Apicidin was not due to contaminant cell sub-populations, but rather to a dynamic equilibrium between cells in different cell-cycle phases or metabolic states.

Genomic DNA was prepared from the two cellular clones showing the best inducibility and low background (clones 1.17 and 2.7), from clones showing constitutive expression of β-lactamase activity (clones 4.5, 5.6, and 3.23), and from a clone showing no background and no inducibility (clone 5.21) using the Qiagen blood and cell culture DNA isolation Kit, Cat. Number: 13362, Qiagen, Valencia, Calif. PCR reactions on genomic DNA templates were performed using primers designed for the amplification of the whole p21m-BLA transcription cassette.

PCR primers and conditions for PCR amplifying the p21m-BlaM transcription cassette were as follows. The forward primer was 5′-CGC GCA CAT TTC CCC GAA AAG TGC-3′ (SEQ ID NO:5) and the reverse primer was 5′-GCA TTT AGG TGA CAC TAT AGA ATA GGG-3′ (SEQ ID NO:6). For the PCR reaction, each reaction contained 2.5 units of Taq DNA polymerase in 1× PCR buffer with MgCl₂ (Roche, Nutley, N.J.; catalog number: 1 596 594), 400 μM dNTPs, 250 nM primers, and 5% DMSO. Reactions were cycled as follows: 96° C. for two minutes, followed by 35 cycles each at 96° C. for 30 seconds, 60° C. for 15 seconds, 72° C. for 60 seconds.

After the above 35 cycles followed by nested PCR using the same primers for an additional 35 cycles as above, DNA fragments of the expected size were amplifiable from the clones 1.17 and 2.7 and the clones with constitutive expression of β-lactamase activity but not from clones without any β-lactamase activity. As shown in FIG. 5, the PCR results show that HDAC inhibitor sensitive clones 2.7 and 1.17 have a lower amplification signal than the cells which constitutively express β-lactamase (clones 4.5, 5.6, and 3.23). This suggests that clones 2.7 and 1.17 have a low copy number of p21m-BLA integrated into the genome whereas the constitutively expressing clones have a high copy number of p21m-BLA integrated into the genome. This further suggests that due to the high sensitivity of the β-lactamase activity assay, only a relatively low copy number of the integrated p21m-BLA reporter gene cassette is compatible with adequate levels of reporter gene transcriptional activity.

### EXAMPLE 4

Clone 1.17 was further characterized by performing dose-response analysis with different classes of known HDAC inhibitors (Apicidin, hydroxamic acids TSA and SAHA, Na-butyrate, and the sulfonamide anilide MS27-275). Apicidin and TSA were tested at concentrations between 1 nM and 10 μM, SAHA between 100 nM and 20 μM, Na-butyrate between 200 μM and 20 mM, MS27-275 between 500 nM and 50 μM. None of the inhibitors tested elicited any induction of Bla activity after a 5 hours stimulus (data not shown). But all five HDAC inhibitors enhanced the reporter transcriptional activity in a dose-dependent manner after about 13 hours treatment. The corresponding EC50 values (means of 3 to 4 independent experiments) for Apicidin, TSA, SAHA, Na-butyrate and MS27-275 were calculated from dose-response data analyzed using a non-linear regression analysis with the aid of KALEIDAGRAPH software (Synergy Software, Reading, Pa.).

Growth inhibition (anti-proliferative) curves were determined after treating clone 1.17 cells for 48 hours with increasing concentrations of the different HDAC inhibitors and the corresponding IC₅₀ values were determined following the CYTOSTAR ¹⁴C-thymidine uptake protocol (Amersham Biosciences, Piscataway, N.J.) described below.

Cells were seeded in a CYTOSTAR-T plate at about 7000 to 10000 cells per well in 100 μL of complete DMEM and incubated 4 hours at 37° C., 5% CO₂. Next, 25 μL per well of HDAC inhibitor or DMSO (vehicle control) 5× solutions in complete DMEM was added to the wells along with 5 μL per well of ¹⁴C-thymidine diluted 1:3 in H₂O (1.65 μL of thymidine per well, 55 mCi/mmol; 225 μCi/mg; 50 μCi/mL (10 μM final concentration) and incubated at 37° C., 5% CO₂. Incorporation of ¹⁴C-thymidine was monitored after 24, 48, and 72 hours by reading the plate in a Packard Instruments TOP-COUNT (CYTOSTAR ¹⁴C method).

Induction of endogenous p21 and acetylation of core histones (inhibition of HDAC activity) was as follows. Dose-response experiments were performed in HeLa cells with different classes of known HDAC inhibitors. Apicidin was tested at concentrations between 37.5 nM and 600 nM, TSA between 100 nM and 800 nM, SAHA between 250 nM and 2 μM, MS27-275 between 200 nM and 200 μM. After 13 hour treatment, whole cell extracts were prepared and analyzed by immunoblotting with a p21-specific antibody (Santa Cruz Biotechnology, Santa Cruz, Calif.; catalog number: sc-397) or an acetylated histone H3-specific antibody (Upstate Biotechnology, Lake Placid, N.Y.; catalog number: 06-599). An α-tubulin-specific antibody (Sigma, St. Louis, Mo.; catalog number: T9026) was used as an internal standard. Quantitative data were obtained by densitometric analysis of X-ray films and were normalized for the internal standard values. The corresponding EC50 values for Apicidin, TSA, SAHA, and MS27-275 were calculated from dose-response data analyzed using a non-linear regression analysis with the aid of KALEIDAGRAPH software. Table 8 summarizes the results.

TABLE 8

| | p21m-Bla | Endogenous p2 | AcH3 | ¹⁴C-thymidine |
|---|---|---|---|---|
| Compound | EC₅₀ (μM) | EC₅₀ (μM) | EC₅₀ (μM) | EC₅₀ (μM) |
| Apicidin | 0.17 | 0.07 | 0.11 | 0.41 |
| SAHA | 1.60 | 0.7 | 0.8 | nd |
| Na-butyrate | 2000 | nd | nd | nd |
| TSA | 0.09 | 0.19 | 0.13 | 0.2 |
| MS27-275 | 12 | 22 | nd | 10 |

None of the HDAC inhibitors tested appeared to elicit any detectable BlaM transcription after about 5 hours exposure to the inhibitors (similar to what reported in the literature for SAHA). In general, under the conditions in this example, induction of detectable BlaM transcription required about 13 hours of exposure to the HDAC inhibitors.

The reversibility of induction of BlaM transcription was also evaluated for the HDAC inhibitor TSA. Clone 1.17 stimulated with 300 or 600 nM TSA (saturating concentrations) for 13 hours showed complete shut down of β-lactamase activity after 48 hours from TSA removal, in good agreement with the published observation that HeLa cells stimulated with saturating amounts of TSA show complete remission from cell cycle arrest after 48 hours (Hoshikawa et al., Exp. Cell Res. 214: 189-197 (1994)). Complete shut down of Bla transcription was not influenced by cell density suggesting that, contrary to what observed for endogenous p21 in HeLa cells, p21 minimal promoter basal activity is not induced by cell over-confluence. These results support the feasibility of subjecting stable transformants such as clone 1.17 to repeated FACS sorting cycles after treatment with saturating TSA concentrations and subsequently after complete reversion of Bla induction in the absence of the HDAC inhibitor. In addition, they suggest the opportunity to perform HDAC RNA interference experiments under conditions of low basal Bla transcription independent of cell density.

The results compare reasonably well indicating that the transcriptional activation of the reporter gene in clone 1.17 upon exposure to a panel of HDAC inhibitors correlates quite faithfully with different physiological cellular responses to the same treatment, including induction of endogenous p21^{WAF1/CIP1} gene expression, the inhibition of HDAC activity, and the anti-proliferative effects. Furthermore, these results are consistent with a large number of published data showing that in transiently transfected cells the proximal region of the p21^{WAF1/CIP1} promoter is necessary and sufficient for its transcriptional activation upon treatment with several HDAC inhibitors.

### EXAMPLE 5

An assay for identifying an HDAC inhibitor using clone 1.17 cells can be performed as follows.

100 μL aliquot of clone 1.17 cells in complete DMEM are plated into each of the wells of a 96 well plate at about 36,000 cells per well for treatment with a plurality of analytes or DMSO control. Serial dilutions of each analyte to be tested are prepared in complete DMEM. After 4 hours, 25 μL of each serial dilution is added to a well containing 1.17 cells. To at least one well of 1.17 cells, DMSO (5%) is added instead of medium containing an analyte. To at least one well of 1.17 cells, 1 μM of Apicidin is added as a positive control. The cells are then grown at 37° C., 5% CO₂ for about 12 to 16 hours. Afterwards, the medium is removed and 100 μL of DMEM without serum and 25 μL of FRET labeled CCF2-AM Bla substrate diluted in the appropriate buffer according to the manufacturer recommendations is added to each of the wells to give a final concentration of 1 μM CCF2-AM. Fluorescence of each well is read at λₑₓ 405/20 λₑₘ 460/40 on a FLUOSTAR (BMG Labtechnologies, Offenburg Germany).

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. A method for identifying an analyte which is a histone deacetylase (HDAC) inhibitor, which comprises:
(a) providing cells which include a reporter gene encoding an enzyme operably linked to a transcription regulatory sequence which includes nucleotide sequences responsive to an HDAC inhibitor selected from the group consisting of Apicidin, Trichostatin A, sodium butyrate, SAHA, and MS27-275 stably integrated into the genome of the cells;
(b) culturing the cells in a medium which includes the analyte and a substrate for the enzyme; and
(c) measuring activity of the enzyme on the substrate wherein an increase in the activity of the enzyme on the substrate indicates that the analyte is an HDAC inhibitor.

2. The method of claim 1 wherein the cells are mammalian cells.

3. The method of claim 1 wherein the cells are human cells.

4. The method of claim 1 wherein the cells are selected from the group consisting of HeLa cells and MCF7 cells.

5. The method of claim 1 wherein the cells are ICLC PD02008.

6. The method of claim 1 wherein the transcription regulatory sequence includes a transcription regulatory sequence of p21^{WAF1/CIP1} which does not include nucleotide sequences responsive to p53.

7. The method of claim 6 wherein the p21WAF1/CIP1 transcription regulatory sequence includes from about nucleotide −183 to nucleotide +25 of the p21^{WAF1/CIP1} transcription regulatory sequence.

8. The method of claim 6 wherein the p21^{WAF1/CIP1} transcription regulatory sequence includes the nucleotide sequence set forth in SEQ ID NO:1.

9. (canceled)

10. The method of claim 1 wherein the reporter gene encodes β-lactamase.

11. The method of claim 10 wherein the substrate for the β-lactamase includes a cephalosporin cleavage site.

12. The method of claim 1 wherein the substrate is labeled with a donor:acceptor fluorophore pair which is capable of fluorescence resonance energy transfer.

13. A cell comprising a reporter gene encoding an enzyme operably linked to a transcription regulatory sequence which includes nucleotide sequences responsive to an HDAC inhibitor selected from the group consisting of Apicidin, Trichostatin A, sodium butyrate, SAHA, and MS27-275 stably integrated into the genome of the cell.

14. The cell of claim 13 wherein the reporter gene is operably linked to a p21^{WAF1/CIP1} transcription regulatory sequence which includes nucleotide sequences responsive to the histone deacetylase (HDAC) inhibitor and does not nucleotide sequences responsive to p53.

15. The cell of claim 13 wherein the cell does not have a functional p53.

16. The cell of claim 13 wherein the cell is selected from the group consisting of HeLa cells and MCF7 cells.

17. The cell of claim 14 wherein the cell is ICLC PD02008.

18. The cell of claim 14 wherein the p21^{WAF1/CIP1} transcription regulatory sequence includes the nucleotide sequence from about nucleotide −183 to nucleotide +25 of the p21^{WAF1/CIP1} transcription regulatory sequence.

19. The cell of claim 14 wherein the p2l^{WAF1/CIP1} transcription regulatory sequence includes the nucleotide sequence set forth in SEQ ID NO:1.

20. The cell of claim 13 wherein the reporter gene encodes β-lactamase.

21. A plasmid comprising a gene encoding β-lactamase operably linked to a transcription regulatory sequence which includes nucleotide sequences responsive to a histone deacetylase (HDAC) inhibitor selected from the group consisting of Apicidin, Trichostatin A, sodium butyrate, SAHA, and MS27-275.

22. The plasmid of claim 21 wherein the gene encoding the β-lactamase is operably linked to a p21^{WAF1/CIP1} transcription regulatory sequence which includes nucleotide sequences responsive to the histone deacetylase (HDAC) inhibitor and does not nucleotide sequences responsive to p53.

23-32. (canceled)
